(19) United States
(12) Patent Application Publication
(10) Pub. No.: US 2025/0127955 A1
(43) Pub. Date: Apr. 24, 2025

(54) MIXED SOLVENT HYDROGEL SYSTEM FOR WOUND HEALING

(71) Applicant: University of Southern California, Los Angeles, CA (US)

(72) Inventor: Mark E. Thompson, Los Angeles, CA (US)

(21) Appl. No.: 18/689,693

(22) PCT Filed: Sep. 27, 2022
(86) PCT No.: PCT/US2022/044941

§ 371 Date: Mar. 6, 2024

Related U.S. Application Data
(60) Provisional application No. 63/249,489, filed on Sep. 28, 2021.

(51) Int. Cl. A61L 26/00

(52) U.S. Cl. CPC A61L 26/008; A61L 26/0014; A61L 26/0066; A61L 26/0076; A61L 2430/34

(57) ABSTRACT

Described herein is a temperature-responsive hydrogel system, and methods and uses thereof.

### CROSS-REFERENCE TO RELATED APPLICATION

This application claims priority under 35 U.S.C. § 119(e) to U.S. Provisional Application No. 63/249,489, filed Sep. 28, 2021, the contents of which are incorporated herein by reference in their entireties.

### TECHNICAL FIELD

The present disclosure relates generally to the field of treating tissue injuries with hydrogel compositions and in particular, for treating burn and other open wounds on the skin.

### BACKGROUND

Approximately 5-20 percent of combat-related casualties during Operation Iraqi Freedom (OIF) and Operation Enduring Freedom (OEF) were severe burn insults. Nuutila, K., et al., Mil. Med. 2018, 184 and Wolf, S. E., et al., Ann. Surg. 2006, 243 (6), 786-795. In general, burns constitute 10 percent of all combat-related injuries to the head and neck regions. Johnson, B. W., et al., J. Oral Maxillofac. Surg. 2015, 73 (1), 106-111. In the Iraq and Afghanistan conflicts, improvised explosive devices (IEDs) were the primary source of injury and accounted for as much as 87 percent of all burns. Lairet, K. F., et al., Prehosp. Emerg. Care 2012, 16 (2), 273-276. The primary cause of death in military operational burn casualties is from infection and gastronomical complications. Primary distinctions of the military burn casualty population is that they are younger, present with a higher injury severities due to blast as the primary cause, and treatment is more-delayed during military operations than civilian type burn injuries.

In the general civilian population, approximately 500,000 burn patients seek medical attention annually in the U.S. of which 40,000 require hospitalization. Latenser, B. A., et al., J Burn Care Res 2007, 28, 635-58. The World Health Organization estimates that there are 11 million burn injuries that occur annually worldwide of which 180,000 are fatal. The majority of these occur in low- and middle-income locations. In the U.S., most burn injuries occur in children from ages 1-16 years of age and in those of working age from 20-59 years of age. The American Burn Association Burn Repository from 2019 reports that flame type burns are responsible for 41% of burn injuries in the U.S. with scalds responsible for 31%. Chemical (3.5%) and electrical burns (3.6%) are much less common. Worldwide, flame related burns are the most common.

Regardless of the cause of the burn injury, the most pronounced effects are the metabolic and inflammatory effects on these individuals and the resultant suppression of the immunological response. Resultantly, infection is the most common cause of morbidity and mortalities. Infection control is even more essential for burn management in the military because pathogens to which service members in current combat operations are exposed are increasingly resistant to antibiotics. Barillo, D. J. et al., Burns 2014, 40 Suppl 1, S24-9. These pathogens are primarily gram-positive bacteria such as methicillin-resistant Staphylococcus aureus (MRSA) and gram-negative bacteria such as Acinetobacter baumannii-calcoaceticus complex, Pseudomonas aeruginosa, and Klebsiella species. Pangli, H. et al., Burns 2019, 45 (7), 1585-1592. These latter pathogens are notable for their increasing resistance to a broad array of antimicrobial agents. Keen, E. F., 3rd, et al., Burns 2010, 36 (6), 819-25 and Albrecht, M. C., et al., J. Am. Coll. Surg. 2006, 203 (4), 546-50. Because immediate evacuation might not always be possible, these types of injuries may have to be managed in a prolonged field care environment, which presents challenges unique to military populations. Further, because the primary source of burn injury is from blast, there is an elevated risk of infection because they are often experience secondary effects that results in the deposition of dirt and debris within the burn wound. Murray, C. K., Journal of Trauma and Acute Care Surgery 2008, 64 (3).

Thus, there is a need for a solution that will cover a large burn area on a patient, within minutes and seal the wound from infection. Currently available spray on hydrogel solutions have problems with clogging the applicator nozzle and when the concentration of the hydrogel is low enough to prevent clogging of the applicator, the sprayed on solution does a poor job of forming a dressing. Disclosed herein, inter alia, are solutions to these and other problems in the art.

### SUMMARY OF THE DISCLOSURE

In one aspect, provided herein is a method for preparing a temperature-sensitive hydrogel for administration to the skin of a subject in need thereof, wherein the method comprising, or consisting essentially of, or yet further consisting of, aerosol mixing (a) a first solution comprising water and (b) a second solution, to form a hydrogel; wherein the second solution comprises an organic solvent and a polymer selected from a poly(N-alkylacrylamide) or a polyvinylpyrolidone copolymer of a first monomer having formula (1) or formula (2) and at least one other monomer that is different than the first monomer:

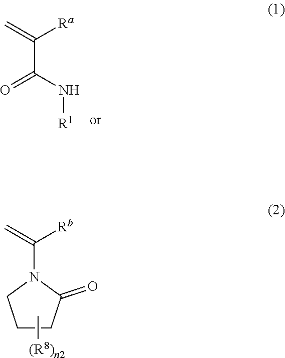

wherein:
R^{a} is H or C₁₋₆ alkyl;
R^{b} is H or C₁₋₆ alkyl;
R¹ is —(CH₂)ₙ₁—R³, C₁₋₆ alkyl, C₆₋₁₈ aryl, or C₄₋₁₈ heteroaryl;
R³ is H, hydroxyl, F, Cl, Br, NH₂, or N(R⁴)₂;
R⁴ is H or C₁₋₆ alkyl;
each R⁸ is independently C₁₋₆ alkyl
n1 is an integer from 0 to 6; and
n2 is 0, 1 or 2.

In embodiments, aerosol mixing occurs initially at ambient temperature, and the temperature of the resulting mixture reduces while evaporating of the organic solvent. In embodiments, the method further comprises aerosol administration of the hydrogel to the skin of the subject after mixing.

In embodiments, the skin comprises a wound.

In embodiments, the aerosol administration of the hydrogel is performed via an aerosol applicator.

In embodiments, the aerosol applicator comprises an electrospinning (ES) applicator, a solution blown spinning (SBS) applicator, a solution blown deposition (SBD) applicator, or a spray deposition (SD) applicator.

In embodiments, the water is present in a mixture of the first solution and the second solution with a weight percentage ranging from about 10% to about 90%, or from about 25% to about 75%.

In embodiments, the water is present in a mixture of the first solution and the second solution with a mass percentage of from about 40% to about 60%.

In embodiments, the polymer is present in the second solution with a concentration of about 0 wt % to about 40 wt %, or about 5 wt % to about 30 wt %.

In embodiments, the polymer is present in the second solution with a concentration of about 10 wt % to about 30 wt %.

In embodiments, the method further comprises evaporating the solvent to form of a fibrous mesh after the aerosol mixing step.

In embodiments, the method further comprises administering a fibrous mesh or a polymer support to the skin of subject where the gel is to be administered.

In embodiments, the gel is deposited on the skin of subject, over the fibrous mesh, or over a polymer support.

In embodiments, the method further comprises reducing temperature of the gel to reduce the adhesive strength of the gel on the skin.

In embodiments, the temperature of the gel is reduced to less than about 15° C.

In embodiments, the temperature of the gel is reduced to less than about 10° C.

In embodiments, the at least one other monomer that is different than the first monomer is described by formula 3a:

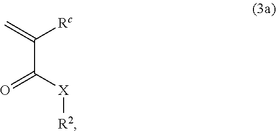

formula 3b:

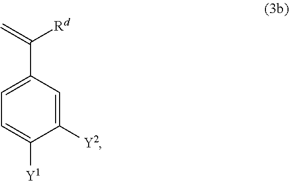

or a combination thereof; wherein
R^{c} is H or C₁₋₆ alkyl;
R^{d} is H or C₁₋₆ alkyl;
R² is H, C₁₋₆ alkyl, C₆₋₁₈ aryl, or C₆₋₁₈ heteroaryl;
X is O or NH; and
Y¹ and Y² are each independently selected from H, C₁₋₆ alkyl, OH, or B(OH)₂.

In embodiments, the organic solvent is selected from ethyl acetate, acetone, ethanol, and any combination of two or more thereof.

In another aspect, provided herein is a temperature-responsive hydrogel system, comprising, or consisting essentially of, or yet further consisting of,
(a) a first solution comprising water, and
(b) a second solution comprising an organic solvent; and
a polymer selected from a poly(N-alkylacrylamide) or a polyvinylpyrolidone copolymer of a first monomer having formula (1) or formula (2) and at least one other monomer that is different than the first monomer:

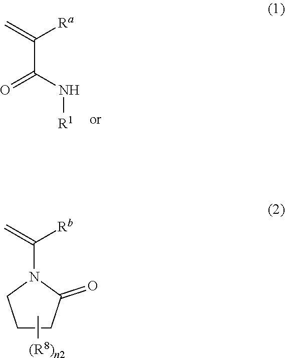

wherein:
R^{a} is H or C₁₋₆ alkyl;
R^{b} is H or C₁₋₆ alkyl;
R¹ is —(CH₂)ₙ₁—R³, C₁₋₆ alkyl, C₆₋₁₈ aryl, or C₆₋₁₈ heteroaryl;
R³ is H, hydroxyl, F, Cl, Br, NH₂, or N(R⁴)₂;
R⁴ is H or C₁₋₆ alkyl;
each R⁸ is independently C₁₋₆ alkyl
n1 is an integer from 0 to 6; and
n2 is 0, 1 or 2.

In embodiments, the polymer is a poly(N-alkylacrylamide) copolymer of a first monomer having formula (1):

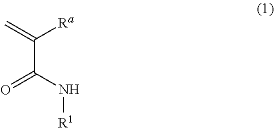

and at least one other monomer that is different than the first monomer.

In embodiments, the polymer is a polyvinylpyrolidone copolymer of a first monomer having formula (2):

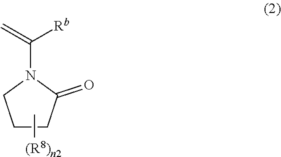

and at least one other monomer that is different than the first monomer.

In embodiments, the at least one other monomer that is different than the first monomer is described by formula 3a:

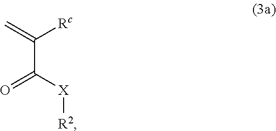

formula 3b:

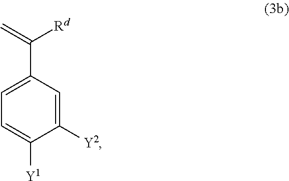

or a combination thereof; wherein
R^{c} is H or C₁₋₆ alkyl;
R^{d} is H or C₁₋₆ alkyl;
R² is H, C₁₋₆ alkyl, C₆₋₁₈ aryl, or C₆₋₁₈ heteroaryl;
X is O or NH; and
Y¹ and Y² are each independently selected from H, C₁₋₆ alkyl, OH, or B(OH)₂.

In embodiments, R¹, R² and R⁸ are each independently methyl, ethyl, n-propyl, iso-propyl, n-butyl, sec-butyl, or tert-butyl.

In embodiments, Y¹ and Y² are both OH.

In embodiments, Y¹ is H or C₁₋₆ alkyl and Y² is B(OH)₂.

In embodiments, Y¹ is B(OH)₂ and Y² is H or C₁₋₆ alkyl.

In embodiments, the organic solvent is selected from ethyl acetate, acetone, ethanol, and any combination of two or more thereof.

In embodiments, the temperature-responsive hydrogel system further comprises a cross-linking agent.

In embodiments, the cross-linking agent is a photo-crosslinking agent.

In embodiments, the photo-crosslinking agent is irradiated by UV light or heat.

In embodiments, the crosslinking agent is a water soluble crosslinking agent.

In embodiments, the crosslinking agent is selected from a polycatechol-containing compound, a guanidine-containing compound or a diol-containing compound.

In embodiments, the crosslinking agent is selected from acrolyl-acetophenone, tannic acid, guanidinopropionic acid, propylene glycol, ethylene glycol diacrylate, ethylene glycol dimethylacrylate, 1,4-dihydrooxybutane dimethacrylate, dethylene glycol dimethyacrylate, propylene glycol dimethacrylate, diethylene glycol dimethacrylate, dipropylene glycol dimethacrylate, diethylene gycol diacrylate, dipropylene glycol diacrylate, divinyl benzene, divinyltoluene, diallyl tartrate, diallyl malate, divinyl tartrate, triallyl melamine, N,N′-methylene bisacryalamide, diallyl maleate, divinyl ether, 1,3-diallyl 2-(2-hydroxyethyl) citrate, vinyl allyl citrate, allyl vinyl maleate, diallyl itaconate, di(2-hydroxyethyl) itaconate, divinyl sulfone, hexahydro-1,3,5-triallyltriazine, triallyl phosphite, diallyl benzenephosphonate, triallyl aconitate, divinyl citraconate, trimethylolpropane trimethacrylate, and diallyl fumarate.

In embodiments, the temperature-responsive hydrogel system further comprises an adhesion-adjusting additive.

In embodiments, the adhesion-adjusting additive is an adhesion-enhancing additive.

In embodiments, a base temperature-responsive hydrogel having the adhesion-enhancing additive has a failure pressure that is at least 2 times greater than a failure pressure for a base temperature-responsive hydrogel having the same composition without the adhesion-enhancing additive. Adhesion can be measured using tension, peel and lap shear tests, adhering to the ASTM standard protocols for each test.

In embodiments, a base temperature-responsive hydrogel having the adhesion-enhancing additive has a failure pressure that is 2 to 6 times greater than a failure pressure for a base temperature-responsive hydrogel having the same composition without the adhesion-enhancing additive. Adhesion can be measured using tension, peel and lap shear tests, adhering to the ASTM standard protocols for each test.

In embodiments, an adhesion-enhancing additive is selected from the group consisting of Arg-Gly-Asp-Ser amino sequence, guanidine-containing compounds, manganese(II) chloride tetrahydrate, and combinations thereof.

In embodiments, the guanidine-containing compounds is selected from the group consisting of aganodine, agmatidine, agmatine, ambazone, amiloride, apraclonidine, aptiganel, argatroban, arginine, argininosuccinic acid, asymmetric dimethylarginine, benexate, benzamil, bethanidine, BIT225, blasticidin s, brostallicin, camostat, cariporide, chlorophenylbiguanide, cimetidine, ciraparantag, creatine, creatine ethyl ester, creatine methyl ester, creatinine, creatinolfosfate, 2-cyanoguanidine, cycloguanil, debrisoquine, dihydrostreptomycin, ditolylguanidine, E-64, ebrotidine, epinastine, eptifibatide, famotidine, glycocyamine, guanabenz, guanadrel, guanazodine, guanethidine, guanfacine, guanidine, guanidine nitrate, guanidinium chloride, guanidinium thiocyanate, 5′-guanidinonaltrindole, 6′-guanidinonaltrindole, guanidinopropionic acid, guanochlor, guanoxabenz, guanoxan, gusperimus, impromidine, kopexil, laninamivir, leonurine, lombricine, lugduname, metformin, methylarginine, mitoguazone, octopine, OUP-16, pentosidine, peramivir, phosphocreatine, picloxydine, pimagedine, polyhexamethylene guanidine, n-propyl-l-arginine, rimeporide, robenidine, saxitoxin, siguazodan, streptomycin, sucrononic acid, sulfaguanidine, synthalin, TAN-1057 A, TAN-1057 C, tegaserod, terbogrel, 1,1,3,3-tetramethylguanidine, tetrodotoxin, tomopenem, triazabicyclodecene, UR-AK49, vargulin, VUF-8430, zanamivir, and combinations thereof.

In embodiments, the adhesion-enhancing additive is 3-guanidinopropionic acid.

In embodiments, the adhesion-enhancing additive is present in an amount of about 0.01 weight percent to about 25 weight percent of the total weight of the temperature-responsive hydrogel.

In embodiments, the adhesion-adjusting additive is a LCST (Lower Critical Solution Temperature)-adjusting additive.

In embodiments, LCST-adjusting additive is polyethylene-glycol (PEG).

In embodiments, a weight percent ratio of N-alkylacrylamide to the at least one other monomer is from about 99:1 to about 50:50, from about 50:1 to about 5:1, from about 20:1 to about 5:1, or from about 10:1 to about 5:1.

In embodiments, the poly(N-alkyacrylamide) copolymer has a number average molecular weight of about 5,000 to about 5,000,000 Daltons.

In embodiments, the poly(N-alkyacrylamide) copolymer has a number average molecular weight of about 10,000 to about 3,000,000 Daltons, about 50,000 to about 200,000 Daltons, about 200,000 to about 500,000 Daltons, about 500,000 to about 1,000,000 Daltons, or about 1,000,000 to about 5,000,000 Daltons.

In embodiments, the poly(N-alkyacrylamide) copolymer is present in an amount of about 0.5 weight percent to about 50 weight percent of the total weight of the temperature-responsive hydrogel.

In embodiments, the poly(N-alkyacrylamide) copolymer is present in an amount of about 10 weight percent to about 60 weight percent of the total weight of the temperature-responsive hydrogel.

In embodiments, the polymer is present in the second solution with a concentration of about 0 wt % to about 40 wt %, about 5 wt % to about 30 wt %, or about 10 wt % to about 30 wt %.

In embodiments, water is present in a mixture of the first solution and the second solution with a mass percentage ranging from about 10% to about 90%, from about 25% to about 75%, or from about 40% to about 60%.

In embodiments, the polyvinylpyrrolidone copolymer is 3-ethyl-1-vinyl-2-pyrrolidone.

In embodiments, the poly(N-alkylacrylamide) copolymer is a copolymer formed from monomers comprising N-isopropylacrylimide and butyl acrylate.

In embodiments, the polyvinylpyrolidone copolymer is a copolymer formed from monomers comprising 2-ethyl-N-vinylpyrrolidone and butyl acrylate.

In embodiments, a copolymer is a block copolymer.

In embodiments, a copolymer is a statistical or random copolymer.

In embodiments, the temperature-responsive hydrogel system further comprises a bioactive agent.

In embodiments, the bioactive agent is selected from silver, a small molecule pharmaceutical, an antibiotic, a chemotherapeutic, an analgesic, an antidepressant, an antiallergenics, an antimicrobial, and an anti-inflammatory compound, optionally contained with a nanoparticle.

In embodiments, the bioactive agent is silver sulfadiazine (SSD), silver nanoparticles, mafenide acetate, polyhexamethylene biguanide (PHMB), bismuth tribromophenate, or any combination or two or more thereof.

In embodiments, release of the bioactive agent is sustained.

In embodiments, the release is sustained over a time period of more than 12 hours, more than 24 hours, more than 48 hours, or more than 72 hours.

In embodiments, the temperature-responsive hydrogel system further comprises one or more additional monomers having formula 4 that are different than the first monomer and second monomer:

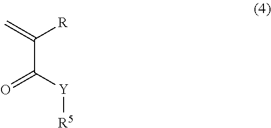

wherein:
Y is O or NR⁶;
R is H or C₁₋₆ alkyl;
R⁵ is —(CH₂)ₘ—R₇;
R⁶ is H or C₁₋₆ alkyl;
R⁷ is halo, hydroxyl, C₆₋₁₂ aryl, C₆₋₁₈ heteroaryl, amino, phosphorylcholinyl, or pyridinyl; and
m is an integer from 0 to 18.

In a related aspect, provided herein is a fibrous mesh or a uniform gel film formed from the temperature-responsive hydrogel system.

In embodiments, the fibrous mesh or the uniform gel film is used for improving wound healing in a subject in need thereof.

In embodiments, the fibrous mesh or the uniform gel film has an adhesive strength sufficient for keeping the fibrous mesh or the uniform gel film in place during subject movement at skin temperature.

In embodiments, the fibrous mesh or the uniform gel film has a reduced adhesive strength upon cooling, compared to the adhesive strength at skin temperature.

In embodiments, the fibrous mesh or the uniform gel film has an adhesive strength of less than about 3 N/cm² or less than about 1 N/cm².

In embodiments, the cooling has a temperature (e.g., temperature of the fibrous mesh or the uniform gel film) of less than about 15° C., or less than about 10° C.

Also provided is a method for preparing a temperature-sensitive hydrogel for administration to the skin of a subject in need thereof, comprising, or consisting essentially of, or yet further consisting of: aerosol mixing (a) a first solution comprising water and (b) a second solution comprising ethanol solvent and a copolymer of N-isopropylacrylamide (NIPAM) and n-butylacrolate (BA), to form a hydrogel; wherein water is present in a mixture of the first solution and the second solution with a weight percentage ranging from 10% to about 90%, about 25% to about 75%, or about 40% to about 60%; the copolymer is present in the second solution with a concentration of about 0 wt % to about 40 wt %, about 5 wt % to about 30 wt %, or about 10 wt % to about 30 wt %.

A temperature-responsive hydrogel system also is disclosed the hydrogel comprising, or consisting essentially of, or yet further consisting of: (a) a first solution comprising water, and (b) a second solution comprising ethanol solvent and a copolymer of N-isopropylacrylamide (NIPAM) and n-butylacrolate (BA); wherein water is present in a mixture of the first solution and the second solution with a weight percentage ranging from 10% to about 90%, about 25% to about 75%, or about 40% to about 60%; the copolymer is present in the second solution with a concentration of about 0 wt % to about 40 wt %, about 5 wt % to about 30 wt %, or about 10 wt % to about 30 wt %.

In another aspect, provided herein is a method for preparing a temperature-responsive or temperature-sensitive hydrogel, comprising, or consisting essentially of, or yet further consisting of aerosol mixing of the first solution and the second solution as described herein, to form a hydrogel. The method can further comprise adding an antibiotic or antimicrobial agent to the first or second solution prior to aerosol mixing of the first and the second solution. In one aspect the antimicrobial comprises silver or a silver nanoparticle.

In another aspect, provided herein is a method for treating the skin of a subject in need thereof comprising, or consisting essentially of, or yet further consisting of applying the temperature-sensitive hydrogel as described herein to the skin of the subject to form a hydrogel, thereby treating the skin of the subject.

In embodiments, the method further comprises, or consists essentially of, or consists of adding an antibiotic or antimicrobial agent to the first or second solution prior to aerosol mixing of the first and the second solution.

In embodiments, the method further comprises, or consists essentially of, or consists of changing the temperature of the hydrogel to release it from the skin of the subject.

In embodiments, the skin of the subject comprises a wound and the temperature sensitive hydrogel is administered to the site of the wound on the subject. Non-limiting examples of wounds include, for example, the wound is selected from an open wound, a cut, a burn, a puncture wound or a bed sore.

The subject to be treated can be a mammal or a human patient.

In another aspect, provided herein is a kit comprising the system as described herein, and instructions for us The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIGS. 1A-1B: (FIG. 1A) Histology (hematoxylin-eosin stain) 48 hours after burn with top-gauze, middle-hydrogel and bottom-no treatment. The edge of the thermal damage is indicated by the arrows. (FIG. 1B) % Epithelialization post treatment.

FIG. 2: Schematic illustration of Applicant's burn dressing, formed from a fibrous mesh and uniform gel film. Both components will be sprayed on from the same applicator.

FIGS. 3A-3B: (FIG. 3A) Schematic of SBS formation of polymer fibers and a commercial airbrush that uses this process (ref. 23b). (FIG. 3B) thick SBS fiber mesh deposited over a hand. Medeiros, E. S., et al., J. Appl. Polym. Sci. (2009).

FIG. 4: This figure illustrates the similarity of human and pig skin.

FIGS. 5A-5B. (FIG. 5A) The monomer structures are illustrated. (FIG. 5B) A crosslinking reaction is illustrated. The dots show cross-linkable units, such as CAT, and the crosses represent crosslinking agents, such as B(OH)₄⁻. After crosslinking the chains are tied into an extended covalently bound network.

FIGS. 6A-6C: Electrospinning of pNI-BA (FIG. 6A) Polymer solution is injected in a high voltage field, charged droplets are pulled to create nanofibers that are collected at the stationary or rotating collector. (FIG. 6B) Electrospinning setup used for generation of pNI-BA nanofibers. The patch size can be increased by using the rotating collector in increasing the size of the collector plate. (FIG. 6C) Electrospun pNI-BA patch separated from the collector.

FIGS. 7A-7D: SBS deposition of pNI-BA in ethanol at low, (FIG. 7A), and high, (FIG. 7B), solution concentrations. (FIG. 7C) shows fibers of pNI-BA prepared by ES. (FIG. 7D) shows sheets formed by mixing water and ethanol in different ratios in SBS deposition. The nominal water content in the film is given, based on the water:pNI-BA ratio in the feed.

FIG. 8: This figure shows monomer structures for UV cross linkable pNI-BA are illustrated.

FIGS. 9A-9B: (FIG. 9A) the catechol based monomer to be used in in situ crosslinking. (FIG. 9B) A crosslinking reaction is illustrated. The green dots show crosslinkable units, such as ADP, and the red crosses represent crosslinking agents, such as B(OH)₄⁻. After crosslinking the chains are tied into an extended covalently bound network.

FIG. 10: Shows the set-up of the apparatus used in Example 7.

FIGS. 11A-11C: The top images (FIG. 11A) show coated substrates (aluminum foil over 10 cm diameter plates). The fluid flow rates and corresponding peristaltic pump rpm values are given for each. A 20% solution of the 98 kD polymer was used, with Air Pressure at 20 psi, Air flow 9.0 SLPM, distance from brush to receiver of 30 cm. The lower images (FIGS. 11B-11C) show the scanning electron microscopy images of the corresponding films in FIG. 11A.

FIG. 12: Lowering the concentration of the polymer in ethanol leads to film formation. The images are for similar deposition conditions as described in Example 7, General Procedure, with a solution flowrate of 2 mL/min. FIG. 12 shows the scanning electron microscopy images of the films obtained using a 10% polymer solution and a 20% polymer solution, respectively. FIG. 12 shows the images of the films obtained using a 5% polymer solution, a 10% polymer solution, and a 20% polymer solution, respectively.

FIG. 13: Ethanol solutions of the polymer were applied to polyurethane substrates using the methods described above (see description for FIG. 11). The strength of adhesion was measured for each film using a standard tension test (adhesive strength is given in Newtons) and the 10% solution of the polymer was shown to give the highest adhesive strength. All of the films were translucent.

FIG. 14: In the films shown in FIG. 14, varying amounts of water were mixed with the ethanol solution of the polymer within the airbrush. The flowrates of the deionized water (DI) and ethanol polymer solution streams were independently controlled to vary the amount of water that was codeposited with the hydrogel polymer. The greatest adhesion is seen for the sample that had roughly equal amounts of water and the ethanol solution of the polymer deposited, shown in the middle (1 mL/min DI, 5 mL/min TRS98-20% EtOH). 1.8, 11 and 9 are the adhesive strength of the hydrogel in Newtons. The y-axis is Newtons. Weight ratio of water to polymer: left (1 mL/min DI, 1 mL/min TRS98-20% EtOH)=5:1, middle (1 mL/min DI, 5 mL/min TRS98-20% EtOH)=1:1, right (1 mL/min DI, 10 mL/min TRS98-20% EtOH)=1:2

### DETAILED DESCRIPTION

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press); PCR 2: A Practical Approach (M. J. MacPherson, B. D. Hames and G. R. Taylor eds., (1995)); Antibodies, a Laboratory Manual, and Animal Cell Culture (R. I. Freshney, ed. (1987)).

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

### Definitions

As used in the specification and claims, the singular form “a,” “an” and “the” include plural references unless the context clearly dictates otherwise. For example, the term “a cell” includes a plurality of cells, including mixtures thereof.

As utilized herein with respect to numerical ranges, the terms “approximately,” “about,” “substantially,” and similar terms will be understood by persons of ordinary skill in the art and will vary to some extent depending upon the context in which it is used. If there are uses of the terms that are not clear to persons of ordinary skill in the art, given the context in which it is used, the terms will be plus or minus 10% of the disclosed values. When “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

As used herein, the singular terms “a,” “an,” and “the” may include plural referents unless the context clearly dictates otherwise. Thus, for example, reference to an object may include multiple objects unless the context clearly dictates otherwise. Alternatively, the use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

In general, “substituted” refers to an alkyl, alkenyl, alkynyl, aryl, or ether group, as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

As used herein, “alkyl” groups include straight chain and branched alkyl groups having from 1 to about 20 carbon atoms, and typically from 1 to 12 carbons or, in some embodiments, from 1 to 8 carbon atoms. As employed herein, “alkyl groups” include cycloalkyl groups as defined below. Alkyl groups may be substituted or unsubstituted. Examples of straight chain alkyl groups include methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups. Representative substituted alkyl groups may be substituted one or more times with, for example, amino, thio, hydroxy, cyano, alkoxy, and/or halo groups such as F, Cl, Br, and I groups. As used herein the term haloalkyl is an alkyl group having one or more halo groups. In some embodiments, haloalkyl refers to a per-haloalkyl group. In some embodiments, the term “alkyl”, as used herein, includes C₁₋₁₂ saturated monovalent hydrocarbon radicals having straight or branched moieties, including, but not limited to, methyl, ethyl, n-propyl, iso-propyl, n-butyl, sec-butyl, tert-butyl, and the like. In some embodiments, alkyl includes C₁₋₆ saturated monovalent hydrocarbon radicals having straight or branched moieties.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups may be substituted or unsubstituted. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to: 2,2-; 2,3-; 2,4-; 2,5-; or 2,6-disubstituted cyclohexyl groups or mono-, di-, or tri-substituted norbornyl or cycloheptyl groups, which may be substituted with, for example, alkyl, alkoxy, amino, thio, hydroxy, cyano, and/or halo groups.

Alkenyl groups are straight chain, branched or cyclic alkyl groups having 2 to about 20 carbon atoms, and further including at least one double bond. In some embodiments alkenyl groups have from 1 to 12 carbons, or, typically, from 1 to 8 carbon atoms. Alkenyl groups may be substituted or unsubstituted. Alkenyl groups include, for instance, vinyl, propenyl, 2-butenyl, 3-butenyl, isobutenyl, cyclohexenyl, cyclopentenyl, cyclohexadienyl, butadienyl, pentadienyl, and hexadienyl groups among others. Alkenyl groups may be substituted similarly to alkyl groups. Divalent alkenyl groups, i.e., alkenyl groups with two points of attachment, include, but are not limited to, CH—CH═CH₂, C═CH₂, or C═CHCH₃.

The term “alkoxy” means a straight or branched-chain alkoxy group. In some embodiments, alkoxy has 1 to 6 carbon atoms (i.e., C₁₋₆ alkoxy). Examples of alkoxy include, but are not limited to, methoxy, ethoxy, propoxy, isopropoxy, butoxy, t-butoxy and the like.

The term alkylalkoxy means a combination of an alkyl or substituted alkyl group and an alkoxy or substituted alkoxy group. In some embodiments, alkylalkoxy has 2 to 10 carbon atoms (i.e., C₂₋₁₀ alkoxy).

As used herein, “aryl”, or “aromatic,” groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-18 carbons, and in others from 6-12 or even 6-10 carbon atoms in the ring portions of the groups. The phrase “aryl groups” includes groups containing fused rings, such as fused aromatic-aliphatic ring systems (e.g., indanyl, tetrahydronaphthyl, and the like). Aryl groups may be substituted or unsubstituted. In some embodiments, the term “aryl” means a C₆₋₁₈ aromatic carbocyclic ring or ring system, which is unsubstituted or substituted by one or more (e.g., 1-3) substituents. Examples of substituents are C₁₋₆ alkyl, hydroxy, C₁₋₆ alkoxy, and halogen. In some embodiments, the aryl group is phenyl or naphthyl.

The term “heteroaryl” means an aromatic heterocyclic ring or ring system, which is unsubstituted or substituted by one or more (e.g., 1-3) substituents. In some embodiments, the term “heteroaryl” means a C₄₋₁₈ aromatic heterocyclic ring or ring system which is unsubstituted or substituted by one or more (e.g., 1-3) substituents. In some embodiments, heteroaryl groups contain 4-12 atoms, and in others from 4-10 or even 4-6 atoms in the ring portions of the groups. Examples of substituents are C₁₋₆ alkyl, hydroxy, C₁₋₆ alkoxy, and halogen. Examples of aromatic heterocyclic rings include, but are not limited to, pyridino, pyrrolo, thienyl, pyrazalo, imidazalo, thiazalo, oxazalo, triazalo, teatrazalo, oxadiazalo, thiadiazolo, benzofuryl, benzothienyl, benzinidazalo, benzotriazalo, quinololyl, isoquinolyl, and indolyl.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

As used herein, and is well-known in the art, the term “hydrogel” refers to a material that comprises polymeric material which has three-dimensional polymer networks (e.g polymer matrix), and can hold water in its polymer matrix (e.g., at least about 10, 20, 30, 40, 50, or 60 percent by weight of water).

As used herein, the term “crosslinker” or “crosslinking agent” refers to an agent that links one entity (e.g., one polymer chain) to another entity (e.g, another polymer chain). For example, a linkage (i.e., the “crosslink”) between two entities can be or can comprise a covalent bond. In some embodiments, a crosslinker may be a small molecule for inducing formation of a covalent bond. In some embodiments, a crosslinker may comprise a photo-sensitive functional group. In some embodiments, a crosslinker may comprise a pH-sensitive functional group. In some embodiments, a crosslinker may comprise a thermal-sensitive functional group.

As used herein, and is well-known in the art, the term “lower critical solution temperature” (LCST) refers to the critical temperature below which the components of a mixture are miscible for all compositions.

As used herein, the term “copolymer” refers to a polymer having more than one type of monomer units. The term “grafted copolymer” refers to a copolymer with a linear backbone of one polymer and randomly distributed side chains of another polymer. The term “block copolymer” refers to a type of copolymer that is made up of blocks of different polymerized monomers. The term “statistical copolymer” refers to a copolymer comprising macromolecules in which the sequential distribution of the monomeric units obeys known statistical laws, including, but not limited to Markovian statistics. The term “random copolymer” refers to a polymeric material that includes at least two different polymeric units (or repeat units) that are covalently bonded to each other in a randomized fashion along the polymer backbone.

As used herein, term “bioactive agent” refers to an agent that is capable of exerting a biological effect in vitro and/or in vivo. The biological effect can be therapeutic in nature.

As used herein, the term “sustained” refers to an extended period of time. For example, “sustained release” broadly refers to the release of a compound from a formulation over an extended or prolonged period of time (e.g., release during 12, 24, 48, 72 or more hours).

As used herein, the term “adhesive strength” refers to the ability of the compositions of the present invention to be able to remain attached to the tissues at the site of administration when subjected to physical stresses or environmental conditions.

As used herein, the term “temperature-responsive” or temperature-sensitive refers to changes in properties (e.g., changes in adhesive strength) of a material with changes in temperature (e.g., the temperature of the material).

Administration or treatment in “combination” refers to administering two agents such that their pharmacological effects are manifest at the same time. Combination does not require administration at the same time or substantially the same time, although combination can include such administrations.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response such as inhibiting a biological target; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.

### Systems

In one aspect, provided herein is a temperature-responsive hydrogel system comprising, or consisting essentially of, or yet further consisting of:
(a) a first solution comprising water, and
(b) a second solution comprising an organic solvent;
   a polymer selected from a poly(N-alkylacrylamide) or a polyvinylpyrolidone copolymer of a first monomer having formula (1) or formula (2) and at least one other monomer that is different than the first monomer:

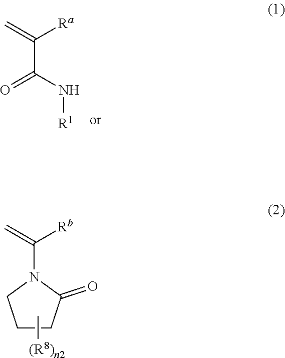

wherein:
R^{a} is H or C₁₋₆ alkyl;
R^{b} is H or C₁₋₆ alkyl;
R¹ is —(CH₂)ₙ₁—R³, C₁₋₆ alkyl, C₆₋₁₈ aryl, or C₆₋₁₈ heteroaryl;
R³ is H, hydroxyl, F, Cl, Br, NH₂, or N(R⁴)₂;
R⁴ is H or C₁₋₆ alkyl;
each R⁸ is independently C₁₋₆ alkyl;
n1 is an integer from 0 to 6; and
n2 is 0, 1, or 2.

In some embodiments, the polymer is a poly(N-alkylacrylamide) copolymer of a first monomer having formula (1):

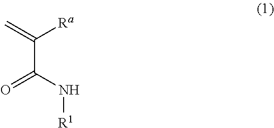

and at least one other monomer that is different than the first monomer.

In some embodiments, the polymer is a polyvinylpyrolidone copolymer of a first monomer having formula (2):

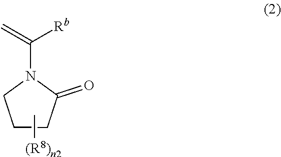

and at least one other monomer that is different than the first monomer.

In some embodiments, the temperature-responsive hydrogel system further comprises, or consists essentially of, or yet further consists of, an adhesion-enhancing additive, the temperature-responsive hydrogel having a failure pressure that is at least 2 times greater than a failure pressure for a base temperature-responsive hydrogel having the same composition without the adhesion-enhancing additive. Adhesion can be measured using tension, peel and lap shear tests, adhering to the ASTM standard protocols for each test.

In some embodiments, the at least one other monomer is described by formula 3a:

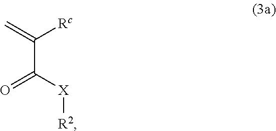

formula 3b:

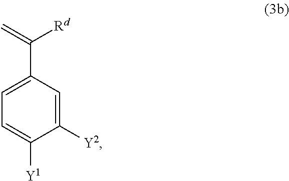

or a combination thereof, wherein
R^{c} is H or C₁₋₆ alkyl;
R^{d} is H or C₁₋₆ alkyl;
R² is H, C₁₋₆ alkyl, C₆₋₁₈ aryl, or C₆₋₁₈ heteroaryl;
X is O or NH; and
Y¹ and Y² are each independently selected from H, C₁₋₆ alkyl, OH, or B(OH)₂.

In some embodiments, the temperature-responsive hydrogel system further comprises, or consists essentially of, or consists of a cross-linking agent selected from a polycatechol-containing compound, a guanidine-containing compound or a diol-containing compound. In some embodiments, the polycatechol-containing compound is tannic acid. In some embodiments, the guanidine-containing compound is guanidinopropionic acid. In some embodiments, the diol-containing compound is propylene glycol.

In some embodiments, the temperature-responsive hydrogel system further comprises, or consists essentially of, or consists of a cross-linking agent selected from ethylene glycol diacrylate, ethylene glycol dimethylacrylate, 1,4-dihydrooxybutane dimethacrylate, dethylene glycol dimethyacrylate, propylene glycol dimethacrylate, diethylene glycol dimethacrylate, dipropylene glycol dimethacrylate, diethylene gycol diacrylate, dipropylene glycol diacrylate, divinyl benzene, divinyltoluene, diallyl tartrate, diallyl malate, divinyl tartrate, triallyl melamine, N,N′-methylene bisacryalamide, diallyl maleate, divinyl ether, 1,3-diallyl 2-(2-hydroxyethyl) citrate, vinyl allyl citrate, allyl vinyl maleate, diallyl itaconate, di(2-hydroxyethyl) itaconate, divinyl sulfone, hexahydro-1,3,5-triallyltriazine, triallyl phosphite, diallyl benzenephosphonate, triallyl aconitate, divinyl citraconate, trimethylolpropane trimethacrylate, and diallyl fumarate.

In some embodiments, the adhesion-enhancing additive is selected from the group of Arg-Gly-Asp-Ser amino sequence, guanidine-containing compounds, manganese(II) chloride tetrahydrate, or combinations thereof.

In some embodiments, the organic solvent is selected from ethyl acetate, acetone, ethanol, or combinations thereof. In some embodiments, the organic solvent is ethyl acetate. In some embodiments, the organic solvent is acetone. In some embodiments, the organic solvent is ethanol.

In some embodiments, R¹ and R² are each independently methyl, ethyl, n-propyl, iso-propyl, n-butyl, sec-butyl, or tert-butyl. In some embodiments, R¹ is methyl, ethyl, n-propyl, iso-propyl, n-butyl, sec-butyl, or tert-butyl. In some embodiments, R² is methyl, ethyl, n-propyl, iso-propyl, n-butyl, sec-butyl, or tert-butyl. In some embodiments, R¹ is iso-propyl. In some embodiments, R² is n-butyl. In some embodiments, R¹ is iso-propyl and R² is n-butyl. In some embodiments, R¹ is tert-butyl. In some embodiments, R² is 2-ethyl-hexyl.

In some embodiments, Y¹ and Y² are both OH. In some embodiments, Y¹ is H or C₁₋₆ alkyl and Y² is B(OH)₂. In some embodiments, Y¹ is B(OH)₂ and Y² is H or C₁₋₆ alkyl.

In some embodiments, n1 is 0. In some embodiments n1 is 1. In some embodiments, n1 is 2. In some embodiments, n1 is 3. In some embodiments, n1 is 4. In some embodiments, n1 is 5. In some embodiments, n1 is 6.

In some embodiments, n2 is 0. In some embodiments n2 is 1. In some embodiments, n2 is 2.

In some embodiments, the temperature-responsive hydrogel has a failure pressure that is 2 to 6 times greater than a failure pressure for a base temperature-responsive hydrogel having the same composition without the adhesion-enhancing additive.

In some embodiments, the guanidine-containing compounds is selected from aganodine, agmatidine, agmatine, ambazone, amiloride, apraclonidine, aptiganel, argatroban, arginine, argininosuccinic acid, asymmetric dimethylarginine, benexate, benzamil, bethanidine, BIT225, blasticidin s, brostallicin, camostat, cariporide, chlorophenylbiguanide, cimetidine, ciraparantag, creatine, creatine ethyl ester, creatine methyl ester, creatinine, creatinolfosfate, 2-cyanoguanidine, cycloguanil, debrisoquine, dihydrostreptomycin, ditolylguanidine, E-64, ebrotidine, epinastine, eptifibatide, famotidine, glycocyamine, guanabenz, guanadrel, guanazodine, guanethidine, guanfacine, guanidine, guanidine nitrate, guanidinium chloride, guanidinium thiocyanate, 5′-guanidinonaltrindole, 6′-guanidinonaltrindole, guanidinopropionic acid, guanochlor, guanoxabenz, guanoxan, gusperimus, impromidine, kopexil, laninamivir, leonurine, lombricine, lugduname, metformin, methylarginine, mitoguazone, octopine, OUP-16, pentosidine, peramivir, phosphocreatine, picloxydine, pimagedine, polyhexamethylene guanidine, n-propyl-l-arginine, rimeporide, robenidine, saxitoxin, siguazodan, streptomycin, sucrononic acid, sulfaguanidine, synthalin, TAN-1057 A, TAN-1057 C, tegaserod, terbogrel, 1,1,3,3-tetramethylguanidine, tetrodotoxin, tomopenem, triazabicyclodecene, UR-AK49, vargulin, VUF-8430, zanamivir, or combinations thereof.

In some embodiments, the adhesion-enhancing additive is 3-guanidinopropionic acid. In some embodiments, the adhesion-enhancing additive is Arg-Gly-Asp-Ser amino sequence. In some embodiments, the adhesion-enhancing additive is manganese(II)) chloride tetrahydrate.

In some embodiments, a weight percent ratio of N-alkylacrylamide to the at least one other monomer is from about 99:1 to about 50:50.

In some embodiments, the poly(N-alkyacrylamide) copolymer has a number average molecular weight of about 5,000 to about 5,000,000 Daltons. In some embodiments, the poly(N-alkyacrylamide) copolymer has a number average molecular weight of about 10,000 to about 3,000,000 Daltons.

In some embodiments, the poly(N-alkyacrylamide) copolymer is present in an amount of about 0.5 weight percent to about 50 weight percent of the total weight of the temperature-responsive hydrogel. In some embodiments, the poly(N-alkyacrylamide) copolymer is present in an amount of about 10 weight percent to about 60 weight percent of the total weight of the temperature-responsive hydrogel.

In some embodiments, the polyvinylpyrrolidone copolymer is 3-ethyl-1-vinyl-2-pyrrolidone.

In some embodiments, the adhesion-enhancing additive is present in an amount of about 0.01 weight percent to about 25 weight percent of the total weight of the temperature-responsive hydrogel.

In some embodiments, the poly(N-alkyacrylamide) copolymer contains poly(N-isopropylacrylamide).

In some embodiments, the poly(N-alkyacrylamide) copolymer is a block copolymer. In some embodiments, the poly(N-isopropylacrylamide) copolymer is a statistical or random copolymer.

In some embodiments, the polyvinylpyrolidone copolymer is a block copolymer. In some embodiments, the polyvinylpyrolidone copolymer is a statistical or random copolymer.

In some embodiments, the temperature-responsive hydrogel system further comprises, or consists essentially of, or consists of a bioactive agent. In some embodiments, the bioactive agent is selected from silver, a small molecule pharmaceutical, an antibiotic, a chemotherapeutic, an analgesic, an antidepressant, an antiallergenics, and an anti-inflammatory compound, optionally contained with a nanoparticle. In some embodiments, the bioactive agent is nanoparticulate silver particles.

In some embodiments, the temperature-responsive hydrogel system further comprises, or consists essentially of, or consists of one or more additional monomers having formula 4 that are different than the first monomer and second monomer:

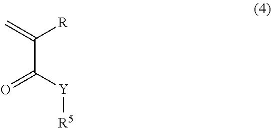

wherein:
Y is O or NR⁶;
R is H or C₁₋₆ alkyl;
R⁵ is —(CH₂)ₘ—R₇;
R⁶ is H or C₁₋₆ alkyl;
R⁷ is halo, hydroxyl, C₆₋₁₂ aryl, C₆₋₁₈ heteroaryl, amino, phosphorylcholinyl, or pyridinyl; and
m is an integer from 0 to 18.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, m is 17. In some embodiments, m is 18.

In some embodiments, the temperature-responsive hydrogel system includes an aerosol applicator.

In some embodiments, the first solution is an electrolyte solution.

In some embodiments, the poly(N-alkylacrylamide) copolymer is a biopolymer. In some embodiments, the poly(N-alkylacrylamide) copolymer is a terpolymer. In some embodiments, the polyvinylpyrolidone copolymer is a biopolymer. In some embodiments, the polyvinylpyrolidone copolymer is a terpolymer.

### Methods

In one aspect, provided herein is a method comprising, or consisting essentially of, or yet further consisting of aerosol mixing the first solution (a) and the second solution (b) as described herein. In embodiments, the mixing occurs (e.g., initially occurs) at ambient temperature. In embodiments, the temperature reduces during mixing due to evaporation the organic solvent in solution (b). In embodiments, the mixing occurs at an effective temperature of less than about 15° C. (e.g., temperature of the resulting mixture).

In some embodiments, the effective temperature (e.g., the temperature of the resulting mixture) is less than about 10° C.

In some embodiments, the method comprises, or consists essentially of, or consists of includes aerosol administration to the skin of a subject in need thereof. In some embodiments, the skin comprises an injury, a wound or an open sore.

In another aspect, provided herein is a method for treating the skin of a subject in need thereof comprising applying the temperature-sensitive hydrogel as described herein to form a hydrogel, thereby treating the subject.

In embodiments, the method further comprises adding an antibiotic or antimicrobial agent to the first or second solution prior to aerosol mixing of the first and the second solution.

In embodiments, the method further comprises changing the temperature of the hydrogel to release it from the skin of the subject.

In embodiments, the skin of the subject comprises a wound and the temperature sensitive hydrogel is administered to the site of the wound on the subject.

In embodiments, the subject is a mammal or a human patient.

In embodiments, the wound is selected from a burn, a puncture or a bed sore.

In another aspect, provided herein is a kit comprising the system as described herein, and instructions for use.

The present disclosure, thus generally described, will be understood more readily by reference to the following examples, which are provided by way of illustration and are not intended to be limiting of the present disclosure.

### EXAMPLES

Burn injuries cause disruptions of the normal skin barrier and impairments of numerous host defense mechanisms that prevent infection. Consequently, until full epithelialization occurs, burn patients remain vulnerable to various invasive microbial infections. Without being bound by theory, Applicant submits that a burn wound can temporized near the point of injury by the application of a thermo-responsive hydrogel adhesive applied by a spray-on procedure for a 24-48 hour duration. To facilitate a simple spray-on application of the dressing, an alcohol solution of the hydrogel is mixed with water at the nozzle of the applicator. The medic need only connect the water and alcohol solutions to the applicator and direct the spray to the burned tissue. The alcohol rich spray solution cools the skin to minimize further injury progression and disinfect the area at the time of application while slowly eluting antibacterial agents from the dressing for a longer duration of coverage. The resultant durable, breathable, hydrogel dressing seals the burn area from foreign containments, maintain a moist environment conducive for healing and be readily removed by irrigation with cold water or a saline solution. The cured hydrogel exhibits a 3D structure which mimics the natural extracellular matrix of skin and its high-water content. The hydrogel dressing is easily and fully removed by cooling the dressing and reapplied via the same spray-on method which eliminates the pain associated with removal of the dressing and limits potential damage to new epithelial growth from traditional gauze bandage removal. Further, a silver nanoparticulate disinfectant. Paladini, F. et al., Materials (2019); Konop, M., et al., Journal of Nanomaterials (2016); and Boonkaew, B., et al., J. Appl. Polym. Sci. (2014) is combined or separately delivered to the burn area from the dressing to minimize or prevent infection.

### Abbreviations

“AM” means acrylamide.
“BA” means butyl acrylate. “PNIPAM” or “pNIPAM” means poly(N-isopropylacrylamide).
“NEAM” means N-ethylacrylamide.
“NMAM” N-methylacrylamide
“NNBAM” means N-n-butylacrylamide.
“NTBAM” means N-t-butylacrylamide.
“TA” means tannic acid.
“VP” means 2-ethyl-N-vinylpyrrolidone.

Applicant developed a medical device for temporary closure of large open globe injuries by military medical personnel with medic training in austere environments.

In one aspect, the polymer is laden with nanoparticulate silver particles which slowly diffuse out of the film to provide antimicrobial activity for the wound. This approach has shown good antimicrobial/antibacterial action in related systems. This hydrogel adhesive dressing enables a medic to spray a free-flowing solution of the hydrogel to the burn, and in minutes result in a secure and breathable wound dressing. Cooling the dressing with cold water or saline leads to loss of adhesion to facilitate ready removal of the dressing. The removal can be performed in the field if necessary.

Ethanol solutions of the pNIPAM-BA hydrogel show high polymer concentration but are not thermo-responsive. Mixed water-ethanol solutions at between 15% and 45% water show comparatively low solubility for the hydrogel at all temperatures. Thus, by mixing a concentrated ethanol solution with pure water or an aqueous electrolyte solution one induces the phase change from a fluid solution to a solid or semisolid hydrogel at as the mixture exits the spray nozzle of an applicator, preventing the clogging that plagues other spray-on applications of hydrogel dressings. The ethanol preferentially evaporates from the spray and deposited solution/suspension leading a cooling of the underlying tissue. This cooling effect and the fact that the hydrogel keeps the burn moist help to promote healing.

An ethanol solution of the hydrogel can be carried into the field without the need for cooling, but the ethanol solution alone will not trigger the phase change needed to form the dressing. Thus, the hydrogel dressing is applied as an aerosol spray, by mixing the ethanol solution of the hydrogel with an aqueous electrolyte solution in the nozzle of the spray applicator, at a fixed water:ethanol ratio that drives gel formation or precipitation of the hydrogel. In one aspect, silver disinfectant particles are dissolved in the aqueous solution (see below). Thus, in this aspect, what is deposited on the tissue is an alcohol/water suspension of the hydrogel and disinfectant. s the alcohol evaporates and the solvent becomes principally water, the hydrogel changes to be between a gel and solid phase. On warming to body temperature, the dressing forms a breathable, solid phase hydrogel, sealing the wound form outside contaminants. This process is slowed by the cooling provided as the alcohol evaporates, but it should be complete in a matter of minutes. This solid phase is ca. 50% water by weight, as expected for a hydrogel. Thus, the medic need only carry the ethanol solution of the hydrogel, the aqueous electrolyte/disinfectant solution and an aerosol applicator to deploy this hydrogel dressing. Thus, at a weight of 2 kg (0.6 kg of dry hydrogel and 1.4 kg for ethanol electrolyte/disinfectant solutions) a medic can prepare 3 L of solution and cover 20-30 sq. ft. of burned tissue assuming a 1-2 mm thick dressing. Cooling the hydrogel dressing releases it from the tissue so that the wound can be cleaned or to carry out more invasive treatment of the wound such as debridement followed by reapplication of the dressing.

### Porcine Skin Studies

This disclosure also provides use of the compositions and methods. Studies carried out on cadaveric porcine skin are used because they are reliable models that closely mimic human wound healing. Compared to other animals, the porcine skin is morphologically and biochemically similar to humans.

This describes in brief the making and use of a uniform “solid” dressing from a mixed water/alcohol spray on porcine skin in 3-5 minutes. The first step is to explore the NIPAM₉BA₁ copolymer hydrogel in a mixed solvent deposition system and embodiments of varying molecular weight of the copolymer, the material compositions (ratio of NIPAM to BA), the solution concentrations and ratios of water to ethanol solution of hydrogel mixed at the spray nozzle that provides a uniform, cohesive thin film/dressing over both healthy and burned tissue are part of this disclosure.

Applicant provides herein a uniform, durable dressing that will show strong temperature dependent adhesion and release on porcine skin. The use of cooling with cold saline irrigation will remove the dressing, but in other aspects, alcohol can be used remove the dressing. Modification of the NIPAM₉BA₁ copolymer can provide varying good adhesion at physiological temperature and release at low temperature as well as being a durable cohesive dressing. The addition of alternate monomers, such as polyvinylpyrolidone (also a thermo-responsive hydrogel) or acrylic acid can be used to impart greater adhesive strength and can readily be incorporated into the synthesis of the copolymer. If the poor durability or cohesive strength of the dressing leads to incomplete removal of the dressing on cooling, the addition of a polymer crosslinker to the aqueous solution is an alternative. In this way, the hydrogel will be crosslinked on application, not prior to initial dissolution in alcohol. Without being bound by theory, the crosslinking is expected to reinforce initial gel formation and help form a uniform thin film dressing over the coated area. Crosslinking can take place at the acrylic acid comonomers.

Spray on Hydrogel Dressing with Antibacterial Properties.

In addition to sealing, hydrating, and protecting the burned tissue, antimicrobial properties can be imparted into the dressing. Silver nanoparticles (NPs) have been used in related wound dressing as an antimicrobial agent for the underlying tissue. Such silver NPs are commercially available from various vendors and can be incorporated into the compositions and methods described herein. See Fortis Life Sciences (https://nanocomposix.com/pages/silver-nanoparticle-safety, last accessed on Sep. 27, 2022 and Sood et al., http://dx.doi.org/10.2174/2213476X05666180614121601, last accessed on Sep. 27, 2022). The NPs are dissolved in the aqueous solution and carried into the dressing as the hydrogel transitions to the gel and solid phases. The pliable hydrogel dressing allows for the silver nanoparticles to slowly diffuse out of the film, providing the desired antimicrobial properties to the dressing. The efficacy of this treatment can be quantified in vitro using cell cultures on NP loaded films with a NP free film of the same material as a reference.

### Burn Progression

The optimal sprayable hydrogel formulations are selected from ex vivo studies, in a complex burn and full thickness porcine model. Deep second degree burns with full thickness injuries incorporated are used. Treatments are applied within 30 minutes to examine the ability of the treatments to reduce the progression of injury. Early assessments are made to histologically determine depth of tissue necrosis and inflammation. The rate of epithelialization and amount of granulation tissue formation also is evaluated.

### Treatment of Wound Infection

Wounds in combat can easily become infected and initial treatment is important. The formulations are assessed on their ability to reduce the bacterial load using a well-established porcine model. Wounds are inoculated with methicillin-resistant Staphylococcus aureus (MRSA) and Pseudomonas aeruginosa, treated and biopsied at various times to determine antimicrobial activity of the treatments. LogCFU/g is used to determined using selective media for each bacterium.

Applicant's spray-on bandages offer broad wound care utility, from burn wound care to serving as an adhesive that can be used to seal small wounds, abrasions, and other surface injuries. The antibiotic agent in the dressing will help prevent infection as well as prevent access of foreign bacteria to the wound. This is applied to the civilian sector. The spray-on burn bandage can have wide applicability to a range of burn injuries. A simple applicator can be used for at-home, EMT and hospital settings to stabilize and treat burns. The bandages can be used beyond the 24-72 hour period targeted since they are readily changed, without the loss of the new tissue. Hydrogels have been shown to beneficial in all stages of wound progression, so this bandage has the potential to not only accelerate healing but decrease scarring as well. Ease of administration and maximal wound care effectiveness minimizes precious resources that would alternatively be diverted from the mission.

### Bandages for Effective Burn Treatment

An optimal dressing for burn wounds must have a number of characteristics: a) provide or maintain moist environment b) enhance epidermal migration c) promote angiogenesis and connective tissue synthesis d) allow gas exchange between wounded tissue and the environment e) maintain appropriate tissue temperature to improve the blood flow to the wound bed and enhances epidermal migration f) provide protection against bacterial infection and g) should be non-adherent to the wound and easy to remove and replace h) provide debridement action to enhance leucocytes migration and support the accumulation of enzyme and i) must be sterile, non-toxic and non-allergic.¹⁴ As shown from data presented herein, hydrogel bandages satisfy many of these desirable properties, including promoting wound healing. In particular the highly hydrated bandage provides excellent hydration of the wound, absorbs exudate, allows for ready gas exchange and provides a cooling effect that helps alleviate pain.¹⁵ However, a property that has not been demonstrated in hydrogel-based burn dressings is adhesion to healthy or burned tissue. On the surface this appears be in line with one of the optimal properties for a burn dressing, i.e. g) the dressing should be non-adherent. This requirement has to do with the need to prevent the removal of new tissue growth on every dressing change, which may be frequent in the early stages of burn healing due to excessive exudate. The bandage proposed here can be switched from an adhesive state, at body temperature, to a totally non-adhesive one by simply cooling the bandage. This makes the removal and changing of the bandage a simple and relatively pain free process that does not remove new tissue. The adhesive properties at body temperature are beneficial for not only keeping the hydrogel in contact with the healing tissue, but in keeping the bandage in place as the patient moves about their space.

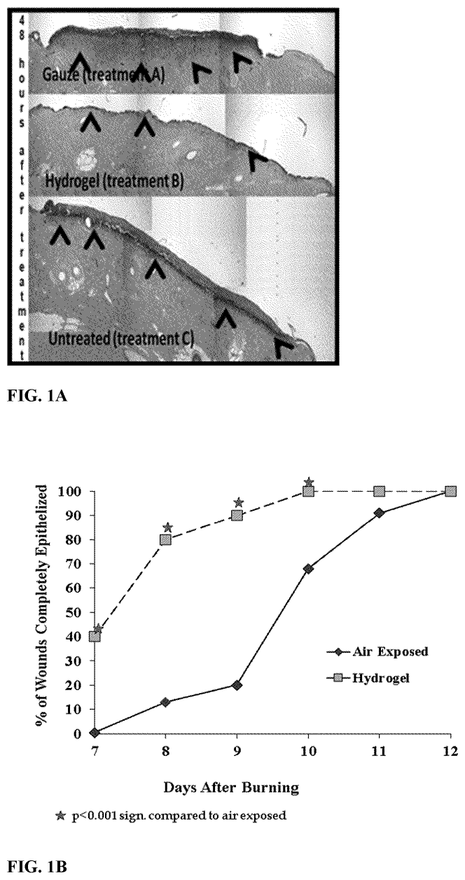

The application of hydrogels has been shown to aid in burn wound progression.¹⁵⁻¹⁶ Wound healing following even an uncomplicated burn injury is a complex process that normally progresses rapidly through an inflammatory phase, followed by a cell proliferation phase and later by a more prolonged wound remodeling phase. The normal progression through these phases is complex in combined injuries such as burns and full thickness injuries. Three characteristic zones of thermal damage have been described following burn injury.¹⁷ The area closest to the thermal insult is the zone of coagulation. This region is not recoverable as all viable structures are destroyed and may consist of only coagulative necrosis and char. The region distal to the zone of coagulation is the zone of stasis, which is functionally impaired but is potentially recoverable. The most outermost region is the zone of hyperemia, which being reactive in nature, typically recovers. As the zone of coagulation is lost and the zone of hyperemia typically recovers, the zone of stasis represents an area where therapeutic intervention may have a significant impact on maximizing the recovery after burn injury. The Miami group has previously observed that hydrogel bandages can reduce burn progression and stimulate epithelization (FIGS. 1A-1B).^{16a,16b} This is due their wound hydration and cooling properties which can reduce the degree of tissue necrosis after 48 hours as compared to untreated or gauze treated wounds (see FIGS. 1A-1B). Other researchers have also described the beneficial properties of hydrogel dressings for promoting recovery form burn injuries.^{15a,16c-16e}

### Thermo-Responsive Hydrogel Bandages for Burn Treatment

In one aspect, the thermo-responsive character described above is provided by the NIPAM component. A homo-polymer of NIPAM shows a transition from the solution phase to solid phase at ca. 30-33° C. This temperature is referred to as the Lower Critical Solution Temperature (LCST). Very few materials show this sort of behavior, i.e. being less soluble at elevated temperature than at low temperature. The BA component in the sealing materials acts to lower the LCST, giving a sharper transition between the solution and solid phases. A polymer composed of 90% NIPAM and 10% BA gives an LCST of 24° C., well below intraocular temperature and skin temperature. Keeping the LCST well below skin temperature of 33° C. is important to maintain adhesion of the bandage. Another polymer that shows the same sort of thermal behavior is poly(2-ethyl-N-vinylpyrrolidone) (pVP), whose LCST is 25° C. In one aspect, Applicant provides hydrogels and methods based on NIPAN and VP, forming copolymers of these thermo-responsive hydrogel monomers and other monomers to control the LCST and promote crosslinking to achieve high durability and stability of the bandage materials.

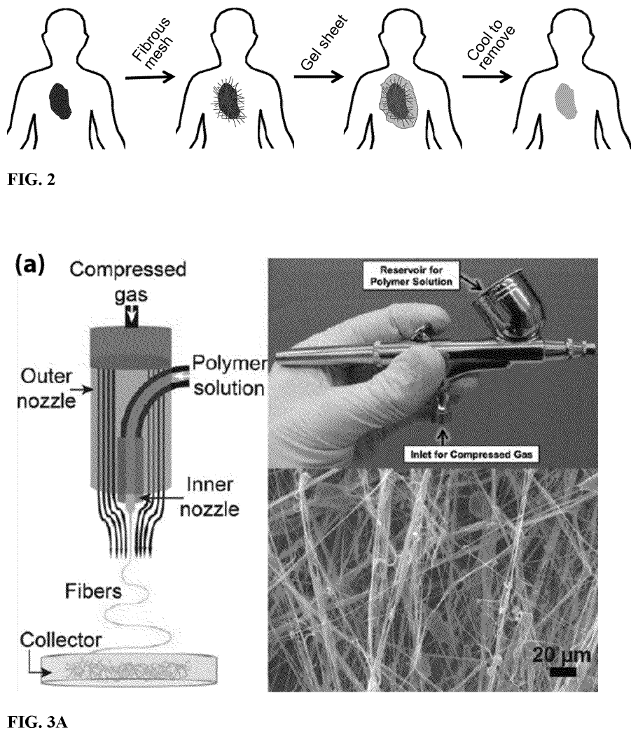

In an alternative embodiment, the bandage can be applied in two parts, the first being a fibrous mesh of the hydrogel and the second a uniform sheet of the same thermo-responsive hydrogel (FIG. 2), both applied in a matter of minutes. The fiber network provides the cohesive strength to make the bandage durable and flexible, while the uniform hydrogel sheet applied over it will provide most of the healing properties of the dressing and provide a medium to load with antibiotics for release into the wound over a period of 24-72 hours.

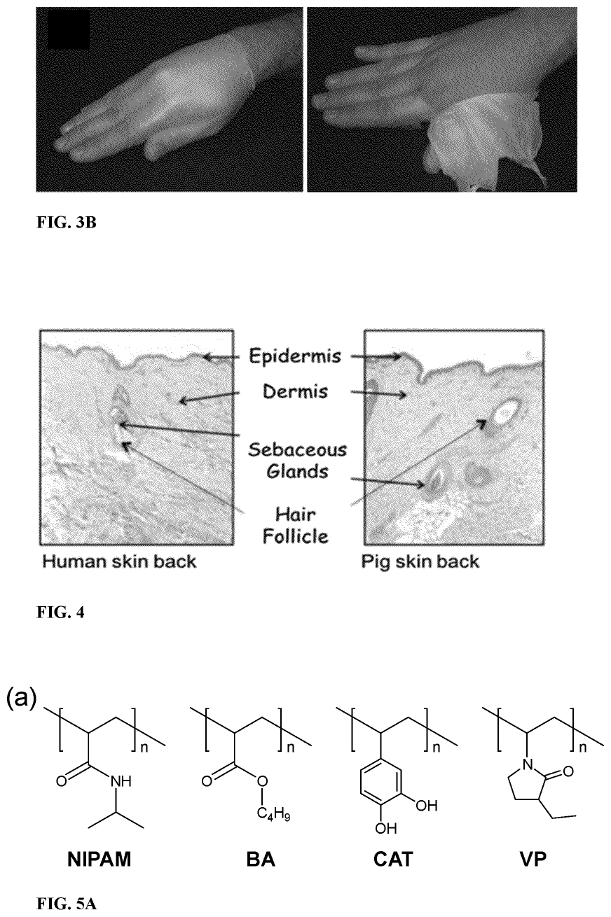

Two methods can be used to produce high quality fibers from polymer solutions of this type, i.e. electrospinning (ES). Xue, J. et al., Chemical Reviews 2019, 119 (8), 5298-5415) and solution blown spinning (SBS); Medeiros, E. S. et al. Journal of Applied Polymer Science 2009, 113 (4), 2322-2330; Daristotle, J. L. et al. ACS Applied Materials & Interfaces 2016, 8 (51), 34951-34963; Gao, Y. at el., Materials Horizons 2021, 8 (2), 426-446.) 10.1039/D0MH01096K. Both of these approaches use a solution of the polymer in a volatile organic solvent. ES uses a high electric field to direct the polymer fiber formation (FIG. 6) and SBS injects the polymer solution into a gas concentric gas stream that evaporates the solvent and forms the fibers (SBS method is further explained below). The ES technique can give highly uniform fibers and form meshes over large areas in good yield. The ES technique requires a high electrical field between the source and substrate that the mesh is deposited on, and may not be used for direct deposition onto a burn wound. In contrast, SBS can be used to deposit the fiber mesh directly onto the burn wound, but it gives less uniform fibers and it has been our experience that to achieve good fiber formation the yield of fibers per volume of solution was low. That being said, SBS polymer fibers have been used to prepare fiber meshes of this type and coat substrates including skin (FIG. 3B). (Medeiros, E. S. et al. Journal of Applied Polymer Science 2009, 113 (4), 2322-2330). The illustration is in FIG. 3A.

A spinning (SBS) and solution blown deposition (SBD) of the organic hydrogel solutions can be used to prepare the porous fiber networks and uniform sheets of the thermo-responsive hydrogels, respectively. Both SBS and SBD use a gas stream to deliver hydrogel fibers and thin films to the wound. The SBS process is illustrated in FIG. 3A. SBS is a well-developed fiber fabrication method that requires parallel concentric gas and fluid streams: a polymer dissolved in a volatile solvent and a pressurized gas that flows around the polymer solution, creating fibers that are deposited in the direction of gas flow.²³ The two streams can be easily integrated into a simple, easy-to use and manufacture device or by using a commercially available airbrush. SBS polymer fibers have been used to prepare fiber meshes of this type and coat substrates including skin (FIG. 3B).²³a The illustrations in FIG. 3B show a single nozzle, but multiple nozzles can be used simultaneously to increase the rate of the fiber mesh deposition.²⁴ To deposit a uniform film, the same sort of gas+solution process described for SBS can be used but it can incorporate a second solution stream. The SBS process relies on the gas stream to evaporate solvent before the stream reaches the substrate. If a second, less volatile solution is added to the liquid stream the less volatile solvent will not evaporate in the gas stream, resulting in the deposition of a concentrated solution of the polymer. This process is referred to as solution blown deposition (SBD). In the case of the hydrogels used here, the polymer can be dissolved in an organic solvent and mixed with water in the nozzle. The organic solvent can evaporate as in SBS and the hydrogel can be left in an aqueous solution stream, which will convert to the hydrated gel/solid on deposition and warming to skin temperature. Prior to evaporation of the organic component it is likely that the polymer will form a gel or solid in the mixed solvent stream,²² however this will not lead to clogging of the nozzle (a common problem with spray on hydrogels^{16e}) since the mixing will take place within the gas stream, after the solution has left the applicator. Antibiotic agents can be added to the organic or aqueous solutions and be incorporated directly into the hydrogel sheet. Slow release of antibiotic agents and other molecular materials from hydrogel dressings is well established,^{16c, 20} and will prevent infection from bacteria on the wound and invasion of external bacteria after the bandage is in place. A spray-on thermo-responsive hydrogel can be used as durable, flexible and breathable bandage for temporizing and treating burn wounds. Further, this bandage can facilitate wound healing and be made antibacterial.

Further, Applicant uses a spray deposition (SD) technique to deposit a uniform film over the mesh. The same applicator used for SBS can be used in spray deposition. If the applicator nozzle is held close to the substrate (ca. 4 inches) or a dilute solution of the polymer is used the solvent does not have sufficient time to fully evaporate and a film is deposited rather than fibers. The SD method has the benefit that it allows us to use mixed solvent systems to vary the hydration level of the hydrogel sheet. This is discussed in the preliminary results given below. We do not expect to need to add an overcoating over the bandage. However, use in a hot, arid environment may lead to partial dehydration of the hydrogel sheet. Occasional treatment with wet gauze will maintain the hydration level of the bandage under these conditions.

Slow release of antibiotic agents and other molecular materials from hydrogel dressings can be established, (Yao, Y. et al., Biomater Sci 2021, 9 (13), 4523-4540; Morsi, N. M. et al., European Journal of Pharmaceutics and Biopharmaceutics 2014, 86 (2), 178-89; and Boonkaew, B. et al., Journal of Pharmaceutical Sciences 2014, 103 (10), 3244-53,) and can prevent infection from bacteria on the wound and invasion of external bacteria after the bandage is in place. The SD method allows for easy loading of the antimicrobial and/or analgesic agent to be incorporated into the deposited thin film.

### Spray-on Hydrogels

The thermo-responsive hydrogel can be applied over a large area as a fibrous network using a simple spray-on method, and a thermo-responsive hydrogel can be applied over a large area as a uniform sheet using a simple spray-on method. In one aspect, the hydrogel formulations and spray methods achieve both a fibrous network and uniform film of a fully hydrated hydrogel, using the same polymer solution for deposition of fibers and films. Without being bound by theory, control and adjustment of the chemical composition of the hydrogel polymer, its molecular weight, polydispersity, concentration in organic and aqueous solutions, as well as the feed rates and ratios of the solutions provides these characteristics.

### Applying a Porous Network of Hydrogel Fibers Over an Area of 2 in².

Applicant provides herein a method for applying a thermo-responsive hydrogel as a uniform film over 2 in² with sufficient cohesion and adhesion to remain in place against gravitational forces and substrate flexure.

Applicant also provides herein a thermo-responsive hydrogel can be applied that will show strong adhesion/cohesion at physiological temperature and full release on cooling. Without being bound by theory, knowledge of the parameters controlling the application of hydrogel fibers and sheets, adjustment of the polymer hydrogel properties in the fibers and sheets can be achieved. Adhesive strengths will be tested and optimized at skin temperature (33° C.) and 10° C. Applicant also provides herein spray-on hydrogels that bind to skin strongly at 33° C. and release on cooling to 10° C. Antibacterial agents can be incorporated into the hydrogel dressing for slow elution into the wound area to prevent bacterial infection of and invasion into the wound. Established antibacterial agents are incorporated into the hydrogel and the properties examined. Elution rates of the antibiotics can be measured for the antibiotic bandages. In vitro antimicrobial studies are conducted to determine optimal formulations to be carried into the in vivo studies. In another aspect, provided herein is a thermo-responsive hydrogel bandage with a sufficiently high loading of an antibacterial agent to show continuous elution of the agent for a period of ≥24 hours. A spray-on hydrogel bandage can positively impact wound progression in 2^{nd} degree burns in a pig model. The key questions are does the bandage remain intact on the freely moving animal and does the dressing promote wound healing of the burn. A well-established pig model is be used. To demonstrate that the optimal formulations can reduce burn progression while stimulating the rate of epithelialization. Performance characteristics of formulations are evaluated.

An antibiotic loaded hydrogel bandages can treat infected wounds and prevent external bacterial infection of the wound. In one aspect, Applicant tests the efficacy of the antibiotic loaded spray-on hydrogel bandages for treating an established biofilm and preventing infection against both gram negative and positive bacteria. Applicant will also demonstrate antimicrobial activity of treatments against both gram negative and positive bacteria.

The application of hydrogels to burn wounds has been shown to aid in wound progression and lead to improved outcomes for burn patients. There are a wide range of hydrogel materials, some derived from naturally occurring materials such as gelatin and collagen as well as artificial ones such as polyethyleneoxide (PEO) and pNIPAM. Polymers formed from NIPAM and 2-ethyl-N-vinylpyrrolidone (VP) are hydrogels with the unique property of being thermo-responsive. These materials show high solubility in cold aqueous solutions (typically <25° C.) and little or no solubility in aqueous solutions at skin temperature (ca. 33° C.). While these polymer hydrogels are insoluble in water at room temperature and above, they are quite soluble in polar organic solvents, such as ethanol and acetone.

At body temperature, the bandage being developed here will form a breathable, solid phase hydrogel, sealing the wound from outside contaminants and providing antibacterial agents to prevent infection. This dressing is ca. 50% water by weight and can take up a significant amount of exudate if it is formed. If the bandage becomes saturated with exudate it can be removed and replaced with a fresh one in a matter of minutes. The medic need only carry the organic solution of the hydrogel, the aqueous electrolyte/disinfectant solution and an applicator with compressed gas cartridges or an air pump to provide the gas source to deploy this hydrogel bandage. Thus, roughly 1 kg of solution will cover 10-15 sq. ft. of burned tissue with a bandage that will be durable and efficiently treat the burn wound. Cooling the hydrogel bandage will release it from the tissue so that the wound can be cleaned or to carry out more invasive treatment of the wound such as debridement followed by reapplication of the bandage. We envision an applicator that will use a simple toggle to choose between the application of fibrous or uniform sheets, both formed from the same organic solution of thermo-responsive hydrogel.

Applicant also proposes two different approaches to coating a wound with a spray-on application of these hydrogels. The first step in forming the bandage is to apply a highly porous, fibrous mat of the hydrogel from the organic solution alone, using solution blown spinning (SBS) techniques.²³ Crosslinking agents are added to the SBS solvent stream to give the fibers good strength and durability. This fibrous network are support for a second hydrogel film, formed of the same hydrogel solution. To apply the second, uniform coating of the thermo-responsive hydrogel Applicant use a mixed solvent aerosol spray. The mixed organic/aqueous medium will promote the gelation and precipitation of the hydrogel²² in the aerosol to give a uniform coating of the hydrogel on the wound and surrounding skin. If the combination of the two films does not have sufficient cohesive strength, crosslinking agents are incorporated into the uniform film as well. Applicant also assesses the durability of the hydrogel sheets and test the adhesion of the bandage to skin at body and low temperatures. The deposition conditions and polymer composition are varied as needed to attain strong adhesion at body temperature and complete release on cooling to 10° C. Applicant also can incorporate antibacterial agents into the hydrogel bandage and reformulate them as needed to maintain their desirable adhesive and mechanical properties while loaded with antibacterial agents that will be released into the wound over a period of 24-72 hours to prevent infection of the wound.

### Example 1

Both Electrospinning (“ES”) and Solution Blow Spinning (“SBS”) are well suited to preparing a fibrous mesh of our thermo-responsive hydrogels. The use of relatively volatile solvents is important to form good quality fibers. Fortunately, the thermo-responsive hydrogel polymers as used herein are quite soluble in solvents such as ethanol and acetone. In order to ensure the solvent has evaporated and the fiber is fully formed the SBS nozzle is typically held 12-15 inches from the substrate being coated. Rastering the SBS source across the surface leads to the deposition of a fiber mesh on the substrate. The parameters that control the size and quality of the fibers produced by SBS include the gas pressure and flow rate, the polymer molecular weight, the polymer concentration, the solvent used in process, the size of the orifice the solution is fed through, the feed rate of the polymer solution into the gas stream and temperature. All of these parameters are easily controlled using a commercial airbrush and a syringe pump or peristaltic pump to control the flow rate of the polymer solution. By careful control of the polymer concentration and feed rate it is possible to prepare fiber meshes where the fibers fuse at the points of contact, making the mesh more stable to deformation. Sanders, E. R. et al. J Vis Exp 2012; Balouiri, M. et al. J Pharm Anal 2016; Beck, N. K. et al., Journal of Rapid Methods & Automation in Microbiology 2009) This may not be needed to form a durable bandage, but it is an option if added stability in the mesh is desired. Once these parameters are optimized for a given polymer they will not change for that polymer and solvent, allowing for a simple applicator with a set of fixed parameters for deposition of the fibrous mesh.

ES has been used to prepare the nanofibers and fiber meshes with a range of hydrogels. Ghosh, T. et al., Polymer Engineering & Science 2021) including NIPAM based hydrogels. Xu, Y. et al., Polymer 2019; Young, R. E. et al., PloS One 2019; Song, M. et al., J Nanosci Nanotechnol 2009; and Wang, J. et al., Soft Matter 2011). Applicant showed in the preliminary results that ES can in fact be used to prepare pNI-BA based meshes. ES allows for better uniformity of the fiber diameter and higher yielding coating of large area substrates than SBS but cannot be directly applied to a wound. The conditions can be modified to electrospin fiber meshes by ES and prepare large area sheets to use in subsequent experiments with the SD films.

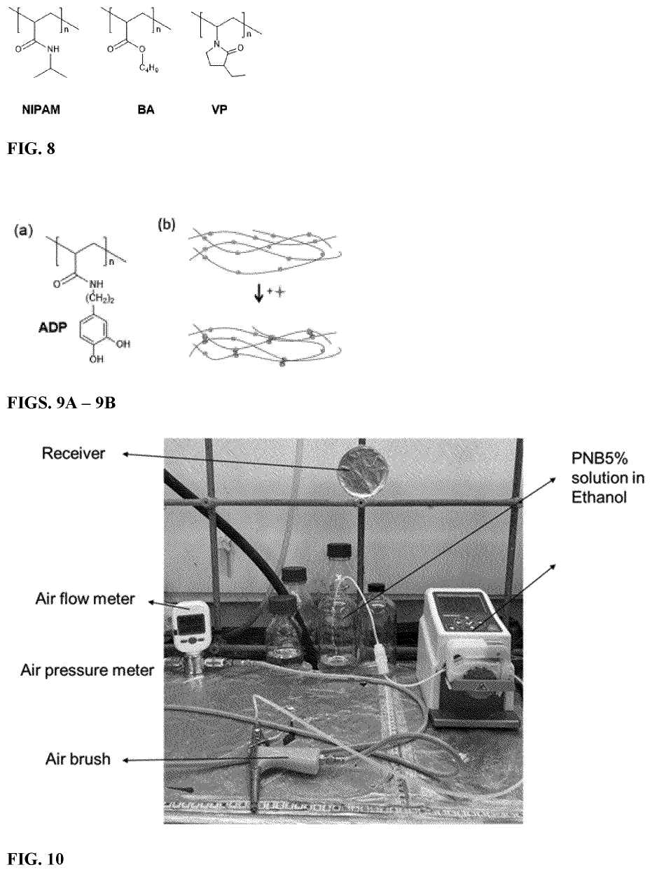

It is important that the fiber mesh not dissolve when the hydrogel sheet is applied by SD. Crosslinking the polymer within the fiber solves this dissolution problem and has been demonstrated for ES grown fibers in general (Ghosh, T. et al., Polymer Engineering & Science 2021) and pNI in particular (Xu, Y. et al., Polymer 2019); Wang, J. et al., Soft Matter 2011). This crosslinking is accomplished by incorporating a photo-crosslinking agent into the fiber and irradiating with UV light or heating to high temperature (160°) after fiber spinning. The UV crosslinking approach involves the use of a NIPAM copolymer with acrolyl-acetophenone (FIG. 8, AP) at between 1 and 5% relative to NIPAM. UV irradiation of the mesh activates the AP groups and crosslinks the polymer strands within the fiber. The UV method will be used here to stabilize the thermo-responsive hydrogel fiber meshes prepared by ES.

The use of UV irradiation or heating to crosslink the fiber mesh applied directly to the burn wound by SBS is not a viable option; the UV irradiation or heating needed to promote crosslinking would injure the patient further and may be problematic to implement in the field. Applicant started the work with SBS formed fibers using the pNI₉BA₁. The studies of SBS and ES described can be modified among a range of molecular weights and solution concentrations for pNI₉BA₁ as well as other compositions of the copolymer, i.e. pNIₓBA₁₋ₓ where x=1-0.5. As the BA concentration is raised, the LCST will drop and adhesion at both body and low temperatures will increase. Importantly, the solubility of the copolymer in water will also drop with an increased BA fraction, making the fibers more stable toward dissolution or softening in the presence of the gel sheet. Decreasing the solubility of the fiber mesh in the solvent system used to deliver the gel sheet may be very important in making a stable and durable bandage. In addition to using the BA content to stabilize the fiber mesh in water, we can use chemical crosslinking to make them stable to dissolution without the need of UV light to promote crosslinking. This approach is described in some detail below for stabilizing thin films, but the same chemistry could be used in the fiber deposition by SBS, albeit with hydrated materials, vide infra.

### Example 2A

Applicant can use SBS formed fibers using the pNIPAM₉BA₁ copolymer that has been used as an ocular sealant. This material can be deposited from an acetone solution using a commercial airbrush with a 0.5 mm orifice and nitrogen carrier gas. Optical and scanning electron microscopies can be used to evaluate the deposited meshes. With this polymer, the ranges in each of the parameters listed above that lead to good fiber formation with a thermo-responsive hydrogel are determined. A range of molecular weights and solution concentrations for pNIPAM₉BA₁ as well as other compositions of the copolymer, i.e. pNIPAMₓBA₁₋ₓ where x=1-0.5 can be evaluated. As the BA concentration is raised the LCST will drop and adhesion at both skin and low temperatures will increase. The solubility of the copolymer in water will also drop with an increased BA fraction, making the fibers more stable toward dissolution or softening in the presence of the gel sheet.

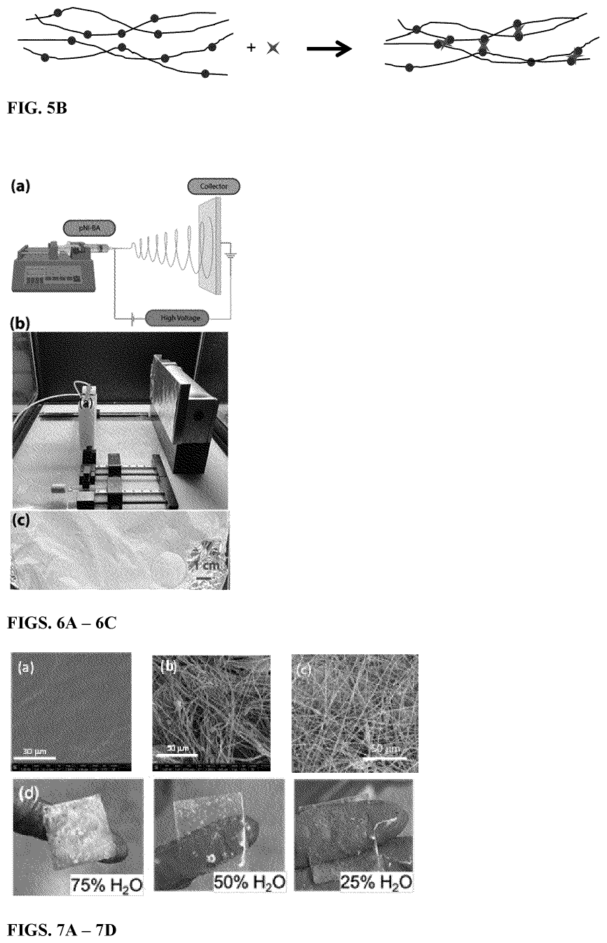

An alternate approach to stabilize the fibers is to use crosslinkers. A crosslinker added during the fiber growth will tie the polymer strands within the fiber together, making the fiber stable to dissolution in organic or aqueous media. We could use the same approach described above for fiber meshes prepared by ES, which involved UV irradiation promoted crosslinking, but there is a simpler way to crosslink SD films that will not require UV irradiation of the film (or the patient). A water soluble crosslinker can be added into the SD process, which will tie the polymer strands together (FIG. 9B). It is important that the crosslinker not be added to the polymer solution itself since this can lead to immediate precipitation and clogging of the applicator nozzle. Having the crosslinker in the water phase will lead to crosslinking taking place as the film is being deposited and not in the initial solution. The key here is to add the crosslinker in the SBS process. Two organic solution streams can be injected into the gas stream, one with the hydrogel polymer and the other a solution of the crosslinker. In this way the crosslinking will take place as the fiber is being formed and not in the initial solution. Applicant has found that tannic acid has a high affinity for pNIPAM based polymers.^{19a} Tannic acid is a multifunctional material that can associate with several polymer chains simultaneously, and will be examined as a crosslinking agent for NIPAM based polymers. The tannic acid interactions with pNIPAM involve hydrogen bonding interactions. A more durable linkage may be needed to give the fibers greater stability. In order to promote a covalent crosslinking process we will add a crosslinkable monomer to the copolymer. Here Applicant relies on the rapid and stable reaction of B(OH)₄⁻ with catechol groups (FIGS. 5A-5B). To use this reaction as a crosslinker, Applicant can incorporate vinyl-catechol (CAT) at a relatively low level in in the polymer pNIPAMₓBA₁₋ₓCAT_{z}. The crosslinking group is typically <8% of the polymer. At this low level the VC will not affect the physical properties of the polymer. Organic solutions of pNIPAMₓBA₁₋ₓCAT_{z} and B(OH)₄⁻ will be mixed in the SBS stream. The two solutions will mix in the SBS gas stream and B(OH)₄⁻ will react rapidly with catechol groups on adjacent polymer chains to form (CAT)B(CAT), crosslinking the polymer (FIG. 5B).

In another aspect, the use of 2-ethyl-N-vinylpyrrolidone (VP) as the thermo-responsive component in the copolymer, i.e. pVPₓBA₁₋ₓ where x=1-0.5 or x=0.9-0.7 is pursued. While the thermo-responsive properties of VP based polymers are well known²¹ their adhesive properties are not.

### Example 2B

In addition to applying a fibrous mesh to the wound and surrounding tissue, a uniform sheet of fully hydrated thermo-responsive hydrogel can be applied. In particular, Applicant will apply a uniform sheet of fully hydrated thermo-responsive hydrogel over a fiber mesh that was deposited by either ES or SBS.

Combining organic and aqueous streams in the spray head will shift the process to SBD. The residual water in the stream will keep the gel hydrated and the lower temperature of the stream afforded by the evaporation of the organic solvent will help ensure that the polymer hydrogel is either in a viscous solution or gel phase when it reaches the skin. Shortly after deposition the body temperature will trigger the formation of the dense hydrogel sheet. As with the fiber deposition by SBS, a number of parameters will control the quality of the film and a detailed study can be pursued to find the optimal ones for the deposition of uniform sheets of the hydrogel.

By adjusting the molecular weight of the polymer, the concentration and the ratio of NIPAM to BA in the polymer we can develop a material that is a cohesive gel at body temperature,^{19b} ideal for both maintaining hydration of the wound and delivering antibacterial agents. If the hydrogel sheet does not show sufficient cohesion to remain bound to the underlying mesh and tissue, such that it will hold its shape with both gravitational forces and the substrate being flexed (simulating the patient moving about) we will incorporate crosslinking agents. Here, the crosslinking groups will be kept at a low fraction of the polymer to prevent rigidifying the sheet.

As Applicant continues, deposition on PET or polyurethane substrates are tested, but Applicant also will advance to artificial skin³⁰ (commercial product: Vitro-Skin®) and cadaveric pig skin as protocols are developed for successful deposition of both the fibrous mesh and uniform sheet forms of the hydrogel.

Both the fibrous mesh and the planar sheet components of the bandage are composed of the same hydrogel polymer, with the principal difference being the level of crosslinking. The same applicator will be used to deposit both films with the operator choosing the solution(s) that are used and selecting a fixed set of parameters for either fiber or film deposition (SBS or SBD). Note that if a mesh prepared by ES is the preferred option this would be used as large, preformed sheets, cut to match the size and shape of the wound and physically applied before overcoating with the hydrogel sheet. We do not envision the medic adjusting the individual parameters, but simply flipping a switch between fiber and sheet depositions. There may need to be an adjustment for the ambient temperature, but this again will be a single adjustment that will change the gas and solution flow rates to the optimal ones for deposition of each type of hydrogel at that temperature. It is expected the sheet to bind strongly to the mesh. If this is not the case, a thin film of crosslinker can be sprayed onto the fibrous mesh before the sheet is applied, to form crosslinks directly between the mesh and sheet.

The proposed bandage consists of two components, a fiber mesh layer for mechanical toughness and a hydrogel sheet to actively treat the burn wound. Our initial studies will focus on using the same polymeric hydrogel for both, with crosslinking agents used to stabilize the fibers to dissolution on deposition of the film. If dissolution of the fiber mesh on SD film deposition proves to be problematic, we will switch to using conventional polymers, such as latex or polyurethane, that form flexible, durable meshes and are totally insoluble in water and ethanol. Both ES and SBS have been used to make fiber meshes with these materials.

### Example 3

With a uniform durable bandage in hand the next step is to measure the adhesion of the bandage to skin at body temperature, typically 33° C., and on lowering the temperature with cold saline irrigation or a cold saline compress. The low temperature here is 5-10° C. In one aspect, Applicant focuses on adhesion studies on artificial skin³⁰ (Vitro-Skin®) and cadaveric pig skin. Applicant will perform tension, peel and lapshear tests, using the ASTM standard protocols for each test (F2258-05, D3330M-04 and F2255-05, respectively). In each case the bandage will be applied by the spray-on methods developed as described above and held at 33-35° C. for 10 minutes before performing the adhesion test. Samples will also be prepared in parallel, “cured” at 33-35° C. for 10 minutes and then cooled by cold saline irrigation or applying a cold compress for different periods of time before performing the adhesion tests. The object or goal is to have an adhesive strength comparable to Tegaderm tape (e.g., ca. 8 N/cm²) at skin temperature and have the adhesion drop by a factor of ten on cooling. The adhesion at low temperature needs to be low enough that the bandage can be washed away or peeled off without disturbing the wound healing process.

Based on Applicant's previous work, Applicant expects that the desired adhesion ranges for warm and cold tissue are reached, however, if the adhesion is not close to our target values, the hydrogel composition to correct for this. If the adhesion level is too low, the BA fraction of the polymer can be increased. pBA and similar polymers are often used as pressure sensitive adhesives in bandages and adhesive strips, with markedly higher adhesive strength than Tegaderm. Increasing the BA fraction in a copolymer both lowers the LCST and increases adhesion level to skin. Adding an acrylic acid component to the polymer will increase adhesion but not significantly affect the LCST. If the adhesion levels for the hydrogel burn bandage are too high, the simple solution is to increase the fraction of NIPAM in the polymer. This will have the added benefit of amplifying the low temperature release of the polymer but will have the negative effect of raising the LCST. If the LCST gets too close to skin temperature the hydrogel sheet component of the bandage will not be mechanically stable, so care must be taken in increasing the NIPAM fraction.

An alternate to increasing the NIPAM content is to add another component to the polymer that is a hydrogel but does not show a thermal response.

If polyethylene-glycol (PEG) is incorporated as a side chain to the hydrogel polymer it will increase the water content in the film and lessen adhesion. The PEG will have the added benefit of providing even greater hydration for the burn.

An alternate to altering the NIPAM content to adjust the LCST is to add another component to the polymer that is a hydrogel but does not show a thermal response. Polyethylene-glycol (PEG) can be codeposited with the thermally responsive hydrogel in the SD process. The added hydrogel will increase the water content in the film at both high and low temperatures and likely lessening the adhesion. The PEG will have the added benefit of providing even greater hydration for the burn.

The use of the fiber mesh and film together is intended to give the bandage sufficient cohesion to make it a durable bandage allowing the patient to move about freely. This is because our experience with NIPAM based polymers is that they demonstrate poor mechanical toughness in their solid form. Crosslinked materials using the SD approach can be prepared. If crosslinked materials prepared by SD have sufficient cohesion and durability to make them an effective bandage without the fiber mesh, it is possible to leave the fiber mesh out of the process, simplifying the application process for the bandages to simply applying a crosslinked hydrogel via SD directly to the burn wound.

### Example 4

Animal studies, rather than artificial skin-like substitutes, are indicated as they will inform us on the durability of SHB on a mobile animal and allow us to directly assess whether the SHB interferes with wound healing. The rodent is chosen as our model system as it is commonly used by other groups, allowing us to compare our results to others. Yu, C. et al., Military Medical Research 2019; Grada, A. et al., Journal of Investigative Dermatology 2018; Ersoy, B. et al., Burns 2016; (d) Otani, N. et al., Annals of Plastic Surgery 2022; Rahmanian-Schwarz, A. et al., Plastic and Reconstructive Surgery 2011. Rats are chosen over mice as larger skin wounds can be generated on the rat.

### Example 5

It is likely that an injured soldier will be treated for their wounds in the field and may not get any more advanced care for 48-72 hours. This makes it imperative to deal with or prevent infection, since if left untreated for 72 hours the infection could cause serious complications and further injury. A common way to apply antibiotics to a large area burn wound is to treat the wound with an ointment, gauze pad or other bandage that is loaded with an antibiotic agent. A number of topical agents have been used in this manner to delay the onset of invasive infection prior to surgical or nonsurgical excision of nonvital tissue. A wide range of antibacterials have been used in these topical burn treatments, ranging from ionic silver to complicated organic molecules. In this program, different antibacterial agents can be used in the hydrogel bandage. These include silver sulfadiazine (SSD)³¹, silver nanoparticles³², mafenide acetate^{33, 34} (PHMB)³⁵ and bismuth tribromophenate³⁶. All of these have been shown to be effective in topical treatments of burns. The antibiotics chosen to study here have a wide range of chemical compositions and structures, which will allow us to probe different antibiotic-hydrogel interaction and see how they affect the properties of the bandage. SSD and mafenide have similar structures and functional groups in the organic component and are both ionic materials. The remaining antibacterials are all neutral and have differing solubilities in water. PHMB is a polymer with average molecular weight of ca. 3000 kD/mol.

The multiple guanidine groups of PHMB will give reasonable water solubility and will interact with the hydrogel leading a slow release. Silver nanoparticles and bismuth tribromophenate will have greater solubility in organic solvents and will likely be segregated into the BA rich regions of the hydrogel bandage. A key issue is which of these antibiotics can be incorporated into the hydrogel bandage without significantly altering the physical properties of the bandage (warm-adhesion and cool-release). Choosing a set of antibacterial agents with a range of chemical properties makes it likely that several of them will work. For all antibacterial agents that do not alter the bandage's adhesion and release behaviors, release of a useful amount of the antibiotic agent to the wound over a 24-72 hour period is ideal. Some of the antibiotics, such as mafenide acetate, have been reported to slow the healing progress in burn wounds, but are very effective at treating infection. Thus, the initial treatment may favor one antibiotic and applications 24 or 48 hours after the initial wound it may make sense to switch to bandage with another antibiotic.

In Applicant's spray-on hydrogel bandage, a similar approach to applying antibiotics to the wound as in the topical approaches referenced above can be used, the antibiotic agent will be eluted out of the hydrogel slowly to provide a constant background level of the agent at the wound. Some of the antibiotics will be better applied via the organic solution of the hydrogel and others from the aqueous solution. The SBD method will concentrate both solutions into a single hydrogel stream that will be deposited onto the wound. The antibiotic will be taken up into the hydrogel as it transitions from a solution to a gel or hydrated solid. The range of antibiotics dissolved in either the organic or aqueous solutions used in the SBD formation of the hydrogel sheet can be examined. A key question is how quickly and over how long a period will each of these antibiotics diffuse out of the hydrogel. The goal is to have significant and sustained release of the antibacterial agent for a period of 24-72 hours.

The testing protocol for the antibiotic dressing will involve preparing hydrogel bandages with a chosen antibiotic in either the organic or the aqueous SBD solution. At the outset it is not clear which solution will be most effective for incorporating the antibiotic into the hydrogel film, so both modalities will be examined. The first question to be asked is if loading the hydrogel with the antibiotic compromises the mechanical and temperature dependent adhesive properties of the bandage. It is not believed this will be the case since the hydrogel is heavily hydrated and the antibiotic is present at a low level relative to the hydrogel. Next, the diffusion rate of the antibiotic out of the dressing is tested. This will be done by submerging the loaded hydrogel in a buffered solution held at 30° C. and monitoring the level of antibiotic in the buffer solution as a function of time.

Those bandages loaded with antibiotics that show sustained release of the antibiotic for a minimum of 24 hours will be carried into the examination for testing against methicillin-resistant Staphylococcus aureus and Pseudomonas aeruginosa. The initial target is 24 hour sustained release, largely due to the expectation that the bandage will be changed daily.

Those bandages loaded with antimicrobials that show sustained release of the antimicrobial for a ≥24 hours will be tested in vitro against multiple strains of Gram-negative, Escherichia coli and Klebsiella pneumoniae, and Gram-positive, methicillin-resistant Staphylococcus aureus and Bacillus thuringiensis, bacteria. Initial antimicrobial efficacy tests will be performed using standard microbiological laboratory techniques. Sanders, E. R. et al., J Vis Exp 2012; Balouiri, M. et al., J Pharm Anal 2016; Beck, N. K. et al., Journal of Rapid Methods & Automation in Microbiology 2009. There is a chance that the heavily hydrated nature of the bandage may make the drugs highly mobile in the film and accelerate drug release, making it difficult to achieve a uniform release profile over the targeted 24-hour period. If this is the case, we will investigate the use of a second, drug loaded SD film. If the antimicrobial and/or analgesic agents are loaded into a polymer film that has a higher BA content, the film will be more hydrophobic and provide for a slow release of the drug into the thermally responsive hydrogel film to be transported to the wound. By tuning the properties of the second film (applied over the top of the bandage) we can tailor the drug release time to match the desired window. The low temperature release of the bandage should not be affected since the same film, optimized for low temperature release in aim 2, will be the one in contact with the wound and surrounding skin.

### Example 6

One of skill in the art can characterize the effect and safety of Spray-on Hydrogel Antimicrobial Bandage (SHAB) application on wound healing in bacterially contaminated partial thickness skin wounds in adult rats as use as an animal model to personalize the system and methods as described herein for the treatment of the skin and wounds using the methods as disclosed herein.

### Example 7

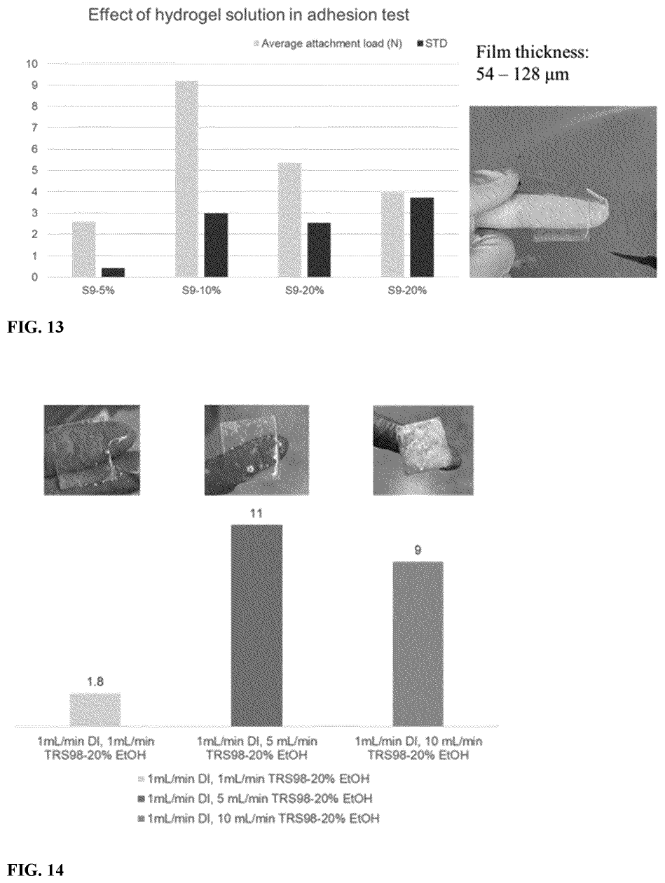

A number of different deposition conditions for solution blown spinning of a copolymer of N-isopropylacrylamide (NIPAM) and n-butylacrolate (BA) were examined (FIGS. 10-14). The ration of NIPAM to BA in this random copolymer was 19:1, respectively. The molecular weight was either 98 kD or 215 kD. Both polymers were readily dissolved in ethanol to levels as high as 30 wt %.

A commercial airbrush was used for the deposition, nitrogen gas was used as the gas carrier, the ethanol solution delivered to the airbrush with a peristaltic pump. Samples were deposited on an aluminum foil target. A range of parameter choices for the deposition were examined.

Synthesis of copolymers. PNIPAM and copolymer of NIPAM and BA (N95BA5) were synthesized using free radical polymerization as reported L. Zou, A. Nair, H. Weng, Y.-T. Tsai, Z. Hu, L. Tang, Intraocular pressure changes: An important determinant of the biocompatibility of intravitreous implants. PLOS ONE 6, e28720 (2011); N. Y. Becerra, et al. Thermosensitive behavior in cell culture media and cytocompatibility of a novel copolymer: Poly(N-isopropylacrylamide-co-butylacrylate). J. Mater. Sci. Mater. Med. 24, 1043-1052 (2013). Using methods described in above references, Applicant was able to synthesize homopolymers and copolymers with various molecular weights and polydispersities.

### General Procedure for Depositing Either Fiber Mesh Sheets or Thin Films Via Solution Blown Methods

An ethanol solution of the thermally responsive hydrogel polymer was applied using a solution blown spinning method. A commercial airbrush was used for the deposition (Grex Tritium.TS3 Double Action Pistol Style Trigger Side Feed Airbrush), with a 0.5 mm nozzle. This air brush used an internal mixing configuration, but an external configuration could be equally efficient in this deposition. Nitrogen was used as the carrier gas and was fed to the airbrush at 20 psi, leading to a gas flow rate of 9.0 SLPM. The solution was fed to the airbrush using a peristaltic pump, so the solution flow rate to the airbrush can be precisely controlled. Here the flow rate is described by the revolutions per minute (rpm) of the peristaltic pump or by mL/min. High rpm is a high flow rate. The distance between the airbrush and the substrate was varied between 10 and 30 cm. At low to moderate solution flow rates we obtained a mesh of microfibers of the polymer. As the flow rate was increased, the deposition transitioned from microfibers to a uniform thin film. As the flow rate was increased, fiber formation gave way to thin film on deposition.

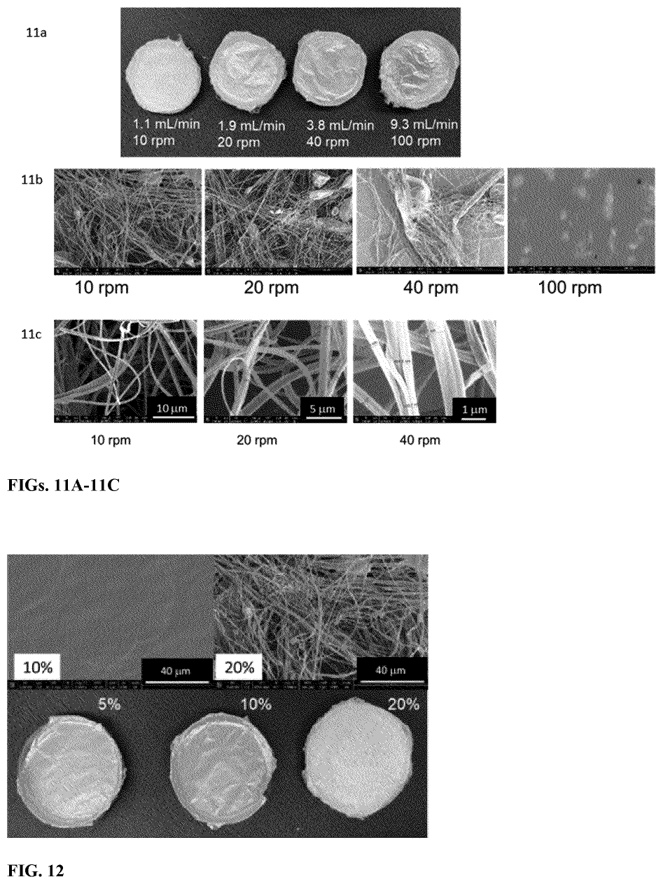

It was found that if the distance between the substrate and the airbrush was decreased from 30 cm to 10 cm, only thin films were formed, i.e. no fibers were observed. The microscopy images of the films formed at close spacing of the substrate and airbrush are similar the film images shown in FIG. 12.

Film adhesion to polyurethane substrates was measured for ethanol solutions of the 98 kD polymer. The adhesive strength was greatest for the 10% solution. This material was a translucent, anhydrous film.

The solution blown spinning method allows for multiple fluid streams to be mixed. We mixed ethanol solutions of the polymer with water in the airbrush. The images in FIG. 14 shows characterizations for TRS98 at 20% in ethanol, gas pressure at 30 psi, gas flowrate 12.0 LPM, distance to the substrate of 10 cm, deposition time was 20 sec. The highest adhesion was observed in the sample that was 50% water by weight, shown in the red bar.

Solutions of the pNI-BA hydrogel were readily deposited by both ES and SBS methods, using commercially available deposition systems. The SBS deposition of an ethanol solution of pNI-BA at low and high concentrations was able to generate uniform sheets and fiber mats of pNI-BA, respectively (FIG. 7A and FIG. 7B). Film deposition was also observed when the more highly concentrated solution was deposited with a short distance between the applicator and the substrate. The SBS deposited film showed strong adhesion to plastic substrates and artificial skin (Vitroskin™). Fibers of the same hydrogel were prepared by ES (FIG. 7C). The fiber mesh prepared by ES had more uniform fiber diameters and was markedly denser than the one prepared by SBS.

While the SD results were encouraging, the ethanol laden polymer did not show the thermo-responsive behavior that is a critical component in our dressing. Using a mixed water-ethanol solution in the applicator led to pNI-BA precipitation, Bischofberger, I. et al., Soft Matter 2014, 10 (41), 8288-8295; Perez-Ramirez, H. A. et al., ACS Applied Polymer Materials 2019, 1 (11), 2961-2972); Hore, M. J. A. et al., Macromolecules 2013; Paladini, F. et al., Materials 2019) clogging the spray nozzles immediately. The SBS method allowed us to use an organic solvent and water as two separate input streams, which effectively mixed at the nozzle of the spray head. The organic solvent would evaporate as in SBS fiber deposition and the hydrogel will be left in a largely aqueous solution stream, which would convert to the hydrated gel/solid on deposition and warming to skin temperature. Prior to evaporation of the organic component it is likely that the polymer would form a gel or solid in the mixed solvent stream, Bischofberger, I. et al., Soft Matter 2014, 10 (41), 8288-8295; Perez-Ramirez, H. A. et al., ACS Applied Polymer Materials 2019, 1 (11), 2961-2972) however this did not lead to clogging of the nozzle since the mixing takes place in the gas stream, after the solution has left the applicator. The three films shown in FIG. 7D were made with mixed ethanol/water feeds to the sprayer and give films that are nominally 25-75% water by weight. These films were hydrogels in this form and show the same thermal transitions that we saw in aqueous solutions of pNI-BA. The film containing 50% water were the most adhesive, showing an adhesive strength similar to that of Tegaderm™.

Provided herein are hydrogel-based spray bandages to address severe burn injuries that in turn, have both short and long-term benefits towards reducing morbidities and mortalities. Severe burn injuries cause disruption to the normal skin barrier and impairments to systemic mechanisms for fluid homeostasis, and thermoregulation. Of primary benefit in the short-term when employing a hydrogel to a burn wound are its hydrophilic properties that enable maintenance of a moist topical environment minimizing additional fluid loss while also providing immediate cooling to prevent further wound conversion of viable tissue from becoming further ischemic. Of secondary importance (after fluid stabilization and reducing further tissue damage) relates to the potential for infection due to the destruction of the skin barrier and the suppressed immunological capability resultant of that systemic anti-inflammatory response that compromise the body's protective mechanisms from infections.

Applicant's hydrogel bandage is an heat-activated hydrogel that transforms into a silicone-like hardened exterior. This provides for a more durable occlusive layer to protect the wound than traditional hydrogel products. The hydrogel's hydrophilic properties enable maintenance of a moist environment and provides a slow natural autolytic debridement process. This results in the sloughing of devitalized tissue and potentially capture the eliminated necrotic tissue and bioburden debris within the hydrogel.

To avert the potential for infection, Applicant's spray bandage provides immediate disinfection of the wound site and in some aspects, can slowly elute antimicrobials and analgesics over a time (e.g., 72-hour duration) to restore immunological defenses while reducing pain associated with nerve cell damage. This lessens the risk of systemic infection that leads to sepsis and multi-organ failure which are the primary cause of fatalities from severe burn wounds that are of most concern in the short-term.

From a long-term perspective, Applicant's hydrogels represent a class of materials that can be widely used in soft tissue engineering of skin, blood vessel, muscle, and fat. Hydrogels are three-dimensional networks consisting of physically or chemically cross-linked bonds of hydrophilic polymers. The insoluble hydrophilic hydrogel's structure demonstrates a remarkable potential to absorb wound exudates and allows oxygen diffusion to accelerate healing. The ability to promote immediate skin regeneration as opposed to longer-term formations of epithelialized scar tissue offers significant long-term esthetic and functional patient benefits. Third and fourth-degree burns can cause severe and extensive disfigurement, nerve damage, and even the loss of a limb. This can be emotionally traumatic and significantly affect the quality of life and possibly the ability to work in the future incurring major financial hardship in the long-term.

Applicant's compositions and methods provides a unique approach to deliver a hydrogel in the form of a spray-on bandage that has never been accomplished in the past due to challenges of atomization that often results in clogging the spray nozzle. In one aspect, Applicant employees an air-gun to ensure maximum rapid coverage of large area wounds with minimal training, minimal preparation, and without increasing the device form factor.

The other primary value of Applicant's methods and compositions is the rapidity that the combination device can be employed. It is critical to treat a burn wound as soon as possible near the time of the injury to avert burn progression to minimize injury severity if possible. Providing a disinfecting spray and a durable moist wound covering provides ideal properties to minimize further tissue damage that will provide significant long-term injury recovery benefits.

And finally, the broad wound utility afforded by Applicant's methods and compositions is that the spray bandage minimizes the assortment of specialty wound care products that need to be stowed in the rucksack in addition to anti-microbial pharma and analgesic creams or pill-packs for which there are recognized oral compliance challenges in severely wounded casualties. A spray bandage that can be administered rapidly via buddy care near the point of injury to address with broad wound care utility while providing prolonged field care durability is anticipated to offer significant impact towards improving outcomes while minimizing resources diverted from the mission.

### EMBODIMENTS

A. A temperature-responsive hydrogel system comprising: (a) a first solution comprising water, and (b) a second solution comprising an organic solvent; and a polymer selected from a poly(N-alkylacrylamide) or a polyvinylpyrolidone copolymer of a first monomer having formula (1) or formula (2) and at least one other monomer that is different than the first monomer:

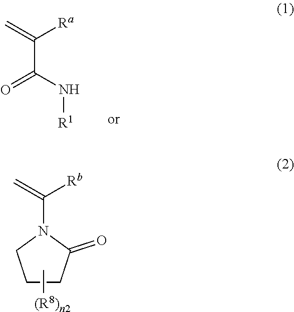

wherein: R^{a} is H or C₁₋₆ alkyl; R^{b} is H or C₁₋₆ alkyl; R¹ is —(CH₂)ₙ₁—R³, C₁₋₆ alkyl, C₆₋₁₈ aryl, or C₆₋₁₈ heteroaryl; R³ is H, hydroxyl, F, Cl, Br, NH₂, or N(R⁴)₂; R⁴ is H or C₁₋₆ alkyl; each R⁸ is independently C₁₋₆ alkyl n1 is an integer from 0 to 6; and n2 is 0, 1 or 2.

B. The temperature-responsive hydrogel system of Paragraph A, wherein the polymer is a poly(N-alkylacrylamide) copolymer of a first monomer having formula (1):

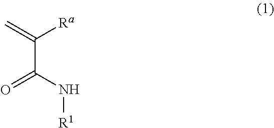

and at least one other monomer that is different than the first monomer.

C. The temperature-responsive hydrogel system of Paragraph A, wherein the polymer is a polyvinylpyrolidone copolymer of a first monomer having formula (2):

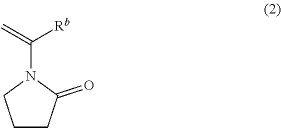

and at least one other monomer that is different than the first monomer.

D. The temperature-responsive hydrogel system of Paragraphs A to C, further comprising an adhesion-enhancing additive, the temperature-responsive hydrogel having a failure pressure that is at least 2 times greater than a failure pressure for a base temperature-responsive hydrogel having the same composition without the adhesion-enhancing additive.

E. The temperature-responsive hydrogel system of any one of Paragraphs A to D, wherein the at least one other monomer is described by formula 3a:

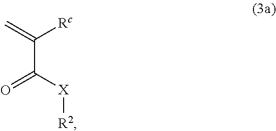

formula 3b:

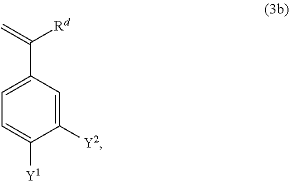

or a combination thereof, wherein

R^{c} is H or C₁₋₆ alkyl; R^{d} is H or C₁₋₆ alkyl; R² is H, C₁₋₆ alkyl, C₆₋₁₈ aryl, or C₄₋₁₈ heteroaryl; X is O or NH; and Y¹ and Y² are each independently selected from H, C₁₋₆ alkyl, OH, or B(OH)₂.

F. The temperature-responsive hydrogel system of any one of Paragraphs A to E, further comprising a cross-linking agent selected from a polycatechol-containing compound, a guanidine-containing compound or a diol-containing compound.

G. The temperature-responsive hydrogel system of any one of Paragraphs A to E, further comprising a cross-linking agent selected from ethylene glycol diacrylate, ethylene glycol dimethylacrylate, 1,4-dihydrooxybutane dimethacrylate, dethylene glycol dimethyacrylate, propylene glycol dimethacrylate, diethylene glycol dimethacrylate, dipropylene glycol dimethacrylate, diethylene gycol diacrylate, dipropylene glycol diacrylate, divinyl benzene, divinyltoluene, diallyl tartrate, diallyl malate, divinyl tartrate, triallyl melamine, N,N′-methylene bisacryalamide, diallyl maleate, divinyl ether, 1,3-diallyl 2-(2-hydroxyethyl) citrate, vinyl allyl citrate, allyl vinyl maleate, diallyl itaconate, di(2-hydroxyethyl) itaconate, divinyl sulfone, hexahydro-1,3,5-triallyltriazine, triallyl phosphite, diallyl benzenephosphonate, triallyl aconitate, divinyl citraconate, trimethylolpropane trimethacrylate, and diallyl fumarate.

H. The temperature-responsive hydrogel system of any one of Paragraphs D to G, wherein the adhesion-enhancing additive is selected from the group consisting of Arg-Gly-Asp-Ser amino sequence, guanidine-containing compounds, manganese(II) chloride tetrahydrate, and combinations thereof.

I. The temperature-responsive hydrogel system of any one of Paragraphs A to H, wherein the organic solvent is selected from ethyl acetate, acetone, ethanol, and combinations thereof.

J. The temperature-responsive hydrogel system of any one of Paragraphs D to I, wherein R¹ and R² are each independently methyl, ethyl, n-propyl, iso-propyl, n-butyl, sec-butyl, or tert-butyl.

K. The temperature-responsive hydrogel system of any one of Paragraphs E to J, wherein Y¹ and Y² are both OH.

L. The temperature-responsive hydrogel system of any one of Paragraphs E to J, wherein Y¹ is H or C₁₋₆ alkyl and Y² is B(OH)₂.

M. The temperature-responsive hydrogel system of any one of Paragraphs E to J, wherein Y¹ is B(OH)₂ and Y² is H or C₁₋₆ alkyl.

N. The temperature-responsive hydrogel system of any previous Paragraph, wherein the temperature-responsive hydrogel having a failure pressure that is 2 to 6 times greater than a failure pressure for a base temperature-responsive hydrogel having the same composition without the adhesion-enhancing additive.

O. The temperature-responsive hydrogel system of any previous Paragraph, wherein the guanidine-containing compounds is selected from the group consisting of aganodine, agmatidine, agmatine, ambazone, amiloride, apraclonidine, aptiganel, argatroban, arginine, argininosuccinic acid, asymmetric dimethylarginine, benexate, benzamil, bethanidine, BIT225, blasticidin s, brostallicin, camostat, cariporide, chlorophenylbiguanide, cimetidine, ciraparantag, creatine, creatine ethyl ester, creatine methyl ester, creatinine, creatinolfosfate, 2-cyanoguanidine, cycloguanil, debrisoquine, dihydrostreptomycin, ditolylguanidine, E-64, ebrotidine, epinastine, eptifibatide, famotidine, glycocyamine, guanabenz, guanadrel, guanazodine, guanethidine, guanfacine, guanidine, guanidine nitrate, guanidinium chloride, guanidinium thiocyanate, 5′-guanidinonaltrindole, 6′-guanidinonaltrindole, guanidinopropionic acid, guanochlor, guanoxabenz, guanoxan, gusperimus, impromidine, kopexil, laninamivir, leonurine, lombricine, lugduname, metformin, methylarginine, mitoguazone, octopine, OUP-16, pentosidine, peramivir, phosphocreatine, picloxydine, pimagedine, polyhexamethylene guanidine, n-propyl-l-arginine, rimeporide, robenidine, saxitoxin, siguazodan, streptomycin, sucrononic acid, sulfaguanidine, synthalin, TAN-1057 A, TAN-1057 C, tegaserod, terbogrel, 1,1,3,3-tetramethylguanidine, tetrodotoxin, tomopenem, triazabicyclodecene, UR-AK49, vargulin, VUF-8430, zanamivir, and combinations thereof.

P. The temperature-responsive hydrogel system of Paragraph O, wherein the adhesion-enhancing additive is 3-guanidinopropionic acid.

Q. The temperature-responsive hydrogel system of any previous Paragraphs, wherein a weight percent ratio of N-alkylacrylamide to the at least one other monomer is from about 99:1 to about 50:50.

R. The temperature-responsive hydrogel system of any previous Paragraphs, wherein the poly(N-alkyacrylamide) copolymer has a number average molecular weight of about 5,000 to about 5,000,000 Daltons.

S. The temperature-responsive hydrogel system of any previous Paragraphs, wherein the poly(N-alkyacrylamide) copolymer has a number average molecular weight of about 10,000 to about 3,000,000 Daltons.

T. The temperature-responsive hydrogel system of any previous Paragraphs, wherein the poly(N-alkyacrylamide) copolymer is present in an amount of about 0.5 weight percent to about 50 weight percent of the total weight of the temperature-responsive hydrogel.

U. The temperature-responsive hydrogel system of any previous Paragraphs, wherein the poly(N-alkyacrylamide) copolymer is present in an amount of about 10 weight percent to about 60 weight percent of the total weight of the temperature-responsive hydrogel.

V. The temperature-responsive hydrogel system of any previous Paragraphs, wherein the polyvinylpyrrolidone copolymer is 3-ethyl-1-vinyl-2-pyrrolidone.

W. The temperature-responsive hydrogel system of any previous Paragraphs, wherein the adhesion-enhancing additive is present in an amount of about 0.01 weight percent to about 25 weight percent of the total weight of the temperature-responsive hydrogel.

X. The temperature-responsive hydrogel system of any previous Paragraphs, wherein the poly(N-alkyacrylamide) copolymer is a block copolymer.

Y. The temperature-responsive hydrogel system of any previous Paragraphs, wherein the poly(N-alkyacrylamide) copolymer is a statistical or random copolymer.

Z. The temperature-responsive hydrogel system of any previous Paragraphs, further comprising a bioactive agent.

AA. The temperature-responsive hydrogel system of Paragraph Z, wherein the bioactive agent is selected from silver, a small molecule pharmaceutical, an antibiotic, a chemotherapeutic, an analgesic, an antidepressant, an antiallergenics, and an anti-inflammatory compound, optionally contained with a nanoparticle.

BB. The temperature-responsive hydrogel system of any prior Paragraphs, further comprising one or more additional monomers having formula 4 that are different than the first monomer and second monomer:

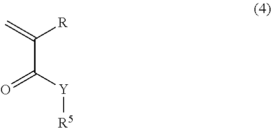

wherein: Y is O or NR⁶; R is H or C₁₋₆ alkyl; R⁵ is —(CH₂)ₘ—R₇; R⁶ is H or C₁₋₆ alkyl; R⁷ is halo, hydroxyl, C₆₋₁₂ aryl, C₆₋₁₈ heteroaryl, amino, phosphorylcholinyl, or pyridinyl; and m is an integer from 0 to 18.

CC. A method comprising aerosol mixing the first solution (a) and the second solution (b) of any of Paragraphs A to BB, at an effective temperature of less than 15° C.

DD. The method of Paragraph CC, wherein the effective temperature is less than 10° C.

EE. The method of Paragraph CC or DD, further comprising aerosol administration to the skin of a subject in need thereof.

FF. The method of Paragraph EE, wherein the skin comprises a wound.

GG. The temperature-responsive hydrogel system of any of Paragraphs A to FF, further comprising an aerosol applicator.

HH. A method for preparing a temperature-sensitive hydrogel for administration to the skin of a subject in need thereof, comprising aerosol mixing (a) a first solution comprising water and (b) a second solution comprising ethanol solvent and a copolymer of N-isopropylacrylamide (NIPAM) and n-butylacrolate (BA), to form a hydrogel; wherein water is present in a mixture of the first solution and the second solution with a weight percentage ranging from 10% to about 90%, about 25% to about 75%, or about 40% to about 60%; the copolymer is present in the second solution with a concentration of about 0 wt % to about 40 wt %, about 5 wt % to about 30 wt %, or about 10 wt % to about 30 wt %.

II. A temperature-responsive hydrogel system comprising: (a) a first solution comprising water, and (b) a second solution comprising ethanol solvent and a copolymer of N-isopropylacrylamide (NIPAM) and n-butylacrolate (BA); wherein water is present in a mixture of the first solution and the second solution with a weight percentage ranging from 10% to about 90%, about 25% to about 75%, or about 40% to about 60%; the copolymer is present in the second solution with a concentration of about 0 wt % to about 40 wt %, about 5 wt % to about 30 wt %, or about 10 wt % to about 30 wt %.

### EQUIVALENTS

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims. Other embodiments are set forth in the following claims.

### REFERENCES

This disclosure makes reference to technical literature by reference to an Arabic number. The full bibliographic citations for these references are provided below.
1. (a) Stoica, A. E.; Chircov, C.; Grumezescu, A. M., Hydrogel Dressings for the Treatment of Burn Wounds: An Up-To-Date Overview. Materials 2020, 13 (12), 2853; (b) Comotto, M., et al., Breathable hydrogel dressings containing natural antioxidants for management of skin disorders. J Biomater. Appl. 2019, 33 (9), 1265-1276.
2. (a) Paladini, F.; Pollini, M., Antimicrobial Silver Nanoparticles for Wound Healing Application: Progress and Future Trends. Materials 2019, 12 (16), 2540; (b) Konop, M., et al., Certain Aspects of Silver and Silver Nanoparticles in Wound Care: A Minireview. Journal of Nanomaterials 2016, 2016, 1-10; (c) Boonkaew, B., et al., Hydrogels containing silver nanoparticles for burn wounds show antimicrobial activity without cytotoxicity. J. Appl. Polym. Sci. 2014, 131 (9).
3. Bayat, N., et al., A reversible thermoresponsive sealant for temporary closure of ocular trauma. Sci. Transl. Med 2017, 9 (419), eaan3879/1-eaan3879/14.
4. (a) Li, B., et al., Reversible Bioadhesives Using Tannic Acid Primed Thermally-Responsive Polymers. Adv. Funct. Mater. 2020, 30 (5), 1907478; (b) Li, B.; Thompson, M. E., Phase transition in amphiphilic poly(N-isopropylacrylamide): controlled gelation. Phys. Chem. Chem. Phys. 2018, 20 (19), 13623-13631; (c) Wahjudi, P. N., et al., Improvement of metal and tissue adhesion on surface-modified parylene C. Journal of Biomedical Materials Research Part A 2009, 89A (1), 206-214.
5. (a) Madaghiele, M., et al., Polymeric hydrogels for burn wound care: Advanced skin wound dressings and regenerative templates. Burns & Trauma 2014, 2 (4); (b) Holzer, J. C. J., et al., A novel human ex-vivo burn model and the local cooling effect of a bacterial nanocellulose-based wound dressing. Burns 2020, 46 (8), 1924-1932; (c) Gil, J., et al., A PEGylated fibrin hydrogel-based antimicrobial wound dressing controls infection without impeding wound healing. International Wound Journal 2017, 14 (6), 1248-1257.
6. (a) Arafa, M. G.; El-Kased, R. F.; Elmazar, M. M., Thermoresponsive gels containing gold nanoparticles as smart antibacterial and wound healing agents. Sci. Rep. 2018, 8 (1), 13674; (b) D′Avignon, L. C., et al., Prevention and Management of Infections Associated With Burns in the Combat Casualty. Journal of Trauma and Acute Care Surgery 2008, 64 (3), S277-S286; (c) Davis, S. C., et al., Preclinical evaluation of a novel silver gelling fiber dressing on Pseudomonas aeruginosa in a porcine wound infection model. Wound Repair Regen. 2019, 27 (4), 360-365; (d) Davis, S. C., et al., The wound-healing effects of a next-generation anti-biofilm silver Hydrofiber wound dressing on deep partial-thickness wounds using a porcine model. International Wound Journal 2018, 15 (5), 834-839.
7. (a) Bischofberger, I.; Calzolari, D. C. E.; Trappe, V., Co-nonsolvency of PNiPAM at the transition between solvation mechanisms. Soft Matter 2014, 10 (41), 8288-8295; (b) Hore, M. J. A., et al., Co-Nonsolvency of Poly(n-isopropylacrylamide) in Deuterated Water/Ethanol Mixtures. Macromolecules 2013, 46 (19), 7894-7901; (c) Perez-Ramirez, H. A.; Haro-Perez, C.; Odriozola, G., Effect of Temperature on the Cononsolvency of Poly(N-isopropylacrylamide) (PNIPAM) in Aqueous 1-Propanol. ACS Applied Polymer Materials 2019, 1 (11), 2961-2972.
8. He, J. J.; McCarthy, C.; Camci-Unal, G., Development of Hydrogel-Based Sprayable Wound Dressings for Second- and Third-Degree Burns. Advanced NanoBiomed Research 2021.
9. (a) Sullivan, T., et al., A New Model for Studying Re-Epithelialization of Partial-Thickness Wounds in Hypoxic Skin. Wounds 2001, 13, 24-28; (b) Meyer, W.; Schwarz, R.; Neurand, K., Skin-Drug Application and Evaluation of Environmental Hazards. Karger Publishers: 1978; (c) Heinrich, W., et al., Isolation and characterization of the large cyanogen bromide peptides from the α1- and α2-chains of pig skin collagen. FEBS Lett. 1971, 16 (1), 63-67; (d) Corr, D. T., et al., Biomechanical behavior of scar tissue and uninjured skin in a porcine model. Wound Repair Regen. 2009, 17 (2), 250-259.
10. (a) Davis, S. C., et al., Antimicrobial effectiveness of wound matrices containing native extracellular matrix with polyhexamethylene biguanide. International Wound Journal n/a (n/a); (b) Cazzaniga, A., et al., The Effect of an Antimicrobial Gauze Dressing Impregnated with 0.2-Percent Polyhexamethylene Biguanide as a Barrier to Prevent Pseudomonas aeruginosa Wound Invasion. Wounds 2002, 14, 169-176; (c) Davis, S. C., et al., Microscopic and physiologic evidence for biofilm-associated wound colonization in vivo. Wound Repair Regen. 2008, 16 (1), 23-29; (d) Davis, S. C., et al., Over-the-counter topical antimicrobials: effective treatments? Archives of Dermatological Research 2005, 297 (5), 190-195; (e) Rodriguez-Menocal, L., et al., Assessment of Ablative Fractional CO2 Laser and Er:YAG Laser to Treat Hypertrophic Scars in a Red Duroc Pig Model. Journal of Burn Care & Research 2018, 39 (6), 954-962; (f) Mertz, P. M., et al., Barrier and Antibacterial Properties of 2-Octyl Cyanoacrylate-Derived Wound Treatment Films. Journal of Cutaneous Medicine and Surgery: Incorporating Medical and Surgical Dermatology 2003, 7 (1), 1-6.
11. (a) Davis, S. C.; Mertz, P. M., Determining the effect of an oak bark formulation on methicillin-resistant Staphylococcus aureus and wound healing in porcine wound models. Ostomy Wound Manage. 2008, 54 (10), 16-8, 20, 22-5; (b) Davis, S. C., et al., Topical oxygen emulsion: a novel wound therapy. Arch. Dermatol. 2007, 143 (10), 1252-6; (c) Davis, S. C.; Mertz, P. M.; Eaglstein, W. H., Second-degree burn healing: the effect of occlusive dressings and a cream. J. Surg. Res. 1990, 48 (3), 245-8.
12. Lairet, J. R., et al., Prehospital interventions performed in a combat zone: A prospective multicenter study of 1,003 combat wounded. Journal of Trauma and Acute Care Surgery 2012, 73 (2), S38-S42.
13. (a) Kopecki, Z., Development of next-generation antimicrobial hydrogel dressing to combat burn wound infection. Biosci. Rep. 2021, 41 (2); (b) Stoica, A. E.; Chircov, C.; Grumezescu, A. M., Hydrogel Dressings for the Treatment of Burn Wounds: An Up-To-Date Overview. Materials (Basel) 2020, 13 (12).
14. (a) Dhivya, S.; Padma, V. V.; Santhini, E., Wound dressings—a review. Biomedicine (Taipei) 2015, 5 (4), 22; (b) Carta, T., et al., Properties of an ideal burn dressing: A survey of burn survivors and front-line burn healthcare providers. Burns 2019, 45 (2), 364-368; (c) Selig, H. F., et al., The properties of an “ideal” burn wound dressing—What do we need in daily clinical practice? Results of a worldwide online survey among burn care specialists. Burns 2012, 38 (7), 960-966.
15. (a) Stoica, A. E.; Chircov, C.; Grumezescu, A. M., Hydrogel Dressings for the Treatment of Burn Wounds: An Up-To-Date Overview. Materials 2020, 13 (12), 2853; (b) Madaghiele, M., et al., Polymeric hydrogels for burn wound care: Advanced skin wound dressings and regenerative templates. Burns & Trauma 2014, 2 (4); (c) Holzer, J. C. J., et al., A novel human ex-vivo burn model and the local cooling effect of a bacterial nanocellulose-based wound dressing. Burns 2020, 46 (8), 1924-1932; (d) Gil, J., et al., A PEGylated fibrin hydrogel-based antimicrobial wound dressing controls infection without impeding wound healing. International Wound Journal 2017, 14 (6), 1248-1257.
16. (a) Gil, J., et al., Hydrogels can reduce dermal temperatures and speed epithelialization of second degree burns. Preliminary evaluations in a porcine model. J. Invest. Dermatol. 2011, 131, 137; (b) Davis, S. C., et al., A new hydrogel dressing accelerates second-degree burn wound healing. J. Invest. Dermatol. 1991, 96, 575; (c) Yao, Y., et al., Recent trends on burn wound care: hydrogel dressings and scaffolds. Biomater Sci 2021, 9 (13), 4523-4540; (d) Tavakoli, S.; Klar, A. S., Advanced Hydrogels as Wound Dressings. Biomolecules 2020, 10 (8); (e) He, J. J.; McCarthy, C.; Camci-Unal, G., Development of Hydrogel-Based Sprayable Wound Dressings for Second- and Third-Degree Burns. Advanced NanoBiomed Research 2021, 1 (6), 2100004.
17. (a) Williams, G., Pathophysiology of the burn wound. In Total Burn Care., 2nd ed.; Herndon, D., Ed. Saunders: 2002; pp 514-22; (b) Jackson, D. M., [The diagnosis of the depth of burning]. Br. J. Surg. 1953, 40 (164), 588-96.
18. Bayat, N., et al., A reversible thermoresponsive sealant for temporary closure of ocular trauma. Sci. Transl. Med. 2017, 9 (419), eaan3879/1-eaan3879/14.
19. (a) Li, B., et al., Reversible Bioadhesives Using Tannic Acid Primed Thermally-Responsive Polymers. Adv. Funct. Mater. 2020, 30 (5), 1907478; (b) Li, B.; Thompson, M. E., Phase transition in amphiphilic poly(N-isopropylacrylamide): controlled gelation. Phys. Chem. Chem. Phys. 2018, 20 (19), 13623-13631; (c) Wahjudi, P. N., et al., Improvement of metal and tissue adhesion on surface-modified parylene C. Journal of Biomedical Materials Research Part A 2009, 89A (1), 206-214.
20. (a) Heskins, M.; Guillet, J. E., Solution Properties of Poly(N-isopropylacrylamide). Journal of Macromolecular Science: Part A—Chemistry 1968, 2 (8), 1441-1455; (b) Schild, H. G.; Tirrell, D. A., Microcalorimetric detection of lower critical solution temperatures in aqueous polymer solutions. The Journal of Physical Chemistry 1990, 94 (10), 4352-4356.
21. (a) Trellenkamp, T.; Ritter, H., 3-Ethylated N-Vinyl-2-pyrrolidone with LCST Properties in Water. Macromol. Rapid Commun. 2009, 30 (20), 1736-40; (b) Lai, H., et al., Thermoresponsive behavior of an LCST-type polymer based on a pyrrolidone structure in aqueous solution. Soft Matter 2012, 8 (9).
22. (a) Bischofberger, I.; Calzolari, D. C. E.; Trappe, V., Co-nonsolvency of PNiPAM at the transition between solvation mechanisms. Soft Matter 2014, 10 (41), 8288-8295; (b) Perez-Ramirez, H. A.; Haro-Perez, C.; Odriozola, G., Effect of Temperature on the Cononsolvency of Poly(N-isopropylacrylamide) (PNIPAM) in Aqueous 1-Propanol. ACS Applied Polymer Materials 2019, 1 (11), 2961-2972.
23. (a) Medeiros, E. S., et al., Solution blow spinning: A new method to produce micro- and nanofibers from polymer solutions. J. Appl. Polym. Sci. 2009, 113 (4), 2322-2330; (b) Daristotle, J. L., et al., A Review of the Fundamental Principles and Applications of Solution Blow Spinning. ACS Applied Materials & Interfaces 2016, 8 (51), 34951-34963; (c) Gao, Y., et al., Recent progress and challenges in solution blow spinning. Materials Horizons 2021, 8 (2), 426-446.
24. (a) Kolbasov, A., et al., Industrial-Scale Solution Blowing of Soy Protein Nanofibers. Ind. Eng. Chem. Res. 2016, 55 (1), 323-333; (b) Jia, C., et al., Mass Production of Ultrafine Fibers by a Versatile Solution Blow Spinning Method. Accounts of Materials Research 2021, 2 (6), 432-446; (c) Jia, C., et al., Highly compressible and anisotropic lamellar ceramic sponges with superior thermal insulation and acoustic absorption performances. Nature Communications 2020, 11 (1), 3732.
25. (a) Morsi, N. M.; Abdelbary, G. A.; Ahmed, M. A., Silver sulfadiazine based cubosome hydrogels for topical treatment of burns: development and in vitro/in vivo characterization. Eur. J Pharm. Biopharm. 2014, 86 (2), 178-89; (b) Boonkaew, B., et al., Development and characterization of a novel, antimicrobial, sterile hydrogel dressing for burn wounds: single-step production with gamma irradiation creates silver nanoparticles and radical polymerization. J. Pharm. Sci. 2014, 103 (10), 3244-53; (c) Kim, M. H., et al., Injectable methylcellulose hydrogel containing silver oxide nanoparticles for burn wound healing. Carbohydr. Polym. 2018, 181, 579-586; (d) Mishra, P. K., et al., Zinc oxide nanoparticles: a promising nanomaterial for biomedical applications. Drug Discovery Today 2017, 22 (12), 1825-1834; (e) Lallo da Silva, B., et al., Relationship Between Structure And Antimicrobial Activity Of Zinc Oxide Nanoparticles: An Overview. International journal of nanomedicine 2019, 14, 9395-9410; (f) Mahamuni-Badiger, P. P., et al., Biofilm formation to inhibition: Role of zinc oxide-based nanoparticles. Mater. Sci. Eng. C Mater. Biol. Appl. 2020, 108, 110319; (g) Hurler, J., et al., Improved burns therapy: liposomes-in-hydrogel delivery system for mupirocin. J. Pharm. Sci. 2012, 101 (10), 3906-15; (h) Zhu, C., et al., A Hydrogel-Based Localized Release of Colistin for Antimicrobial Treatment of Burn Wound Infection. Macromol. Biosci. 2017, 17 (2); (i) Chakavala, S. R., et al., Development and in vivo evaluation of silver sulfadiazine loaded hydrogel consisting polyvinyl alcohol and chitosan for severe burns. J. Pharm. Bioallied Sci. 2012, 4 (Suppl 1), S54-6.
26. Sullivan, T. P., et al., THE PIG AS A MODEL FOR HUMAN WOUND HEALING. Wound Repair Regen. 2001, 9 (2), 66-76.
27. (a) Corr, D. T., et al., Biomechanical behavior of scar tissue and uninjured skin in a porcine model. Wound Repair Regen. 2009, 17 (2), 250-259; (b) Heinrich, W., et al., Isolation and characterization of the large cyanogen bromide peptides from the al- and a2-chains of pig skin collagen. FEBS Lett. 1971, 16 (1), 63-67.
28. (a) Davis, S. C., et al., Preclinical evaluation of a novel silver gelling fiber dressing on Pseudomonas aeruginosa in a porcine wound infection model. Wound Repair Regen. 2019, 27 (4), 360-365; (b) Davis, S. C., et al., The wound-healing effects of a next-generation anti-biofilm silver Hydrofiber wound dressing on deep partial-thickness wounds using a porcine model. International Wound Journal 2018, 15 (5), 834-839; (c) Davis, S. C., et al., Microscopic and physiologic evidence for biofilm-associated wound colonization in vivo. Wound Repair Regen. 2008, 16 (1), 23-29; (d) Mertz, P. M., et al., Barrier and Antibacterial Properties of 2-Octyl Cyanoacrylate-Derived Wound Treatment Films. Journal of Cutaneous Medicine and Surgery: Incorporating Medical and Surgical Dermatology 2003, 7 (1), 1-6; (e) Davis, S. C., et al., The wound-healing effects of a next-generation anti-biofilm silver Hydrofiber wound dressing on deep partial-thickness wounds using a porcine model. Int Wound J 2018, 15 (5), 834-839; (f) Pechter, P. M., et al., Keratin dressings speed epithelialization of deep partial-thickness wounds. Wound Repair Regen. 2012, 20 (2), 236-242; (g) Mertz, P. M.; Oliveira-Gandia, M. F.; Davis, S. C., The Evaluation of a Cadexomer Iodine Wound Dressing on Methicillin Resistant Staphylococcus Aureus (MRSA) in Acute Wounds. Dermatol. Surg. 1999, 25 (2), 89-93.
29. Srinivasan, S., et al., Solution spraying of poly(methyl methacrylate) blends to fabricate microtextured, superoleophobic surfaces. Polymer 2011, 52 (14), 3209-3218.
30. Lir, I.; Haber, M.; Dodiuk-Kenig, H., Skin surface model material as a substrate for adhesion-to-skin testing. J. Adhes. Sci. Technol. 2007, 21 (15), 1497-1512.
31. (a) Norman, G., et al., Antiseptics for burns. Cochrane Database Syst Rev 2017, 7 (7), Cd011821; (b) Heyneman, A., et al., The role of silver sulphadiazine in the conservative treatment of partial thickness burn wounds: A systematic review. Burns 2016, 42 (7), 1377-1386.
32. (a) Gravante, G., et al., Nanocrystalline silver: a systematic review of randomized trials conducted on burned patients and an evidence-based assessment of potential advantages over older silver formulations. Ann. Plast. Surg. 2009, 63 (2), 201-5; (b) Dunn, K.; Edwards-Jones, V., The role of Acticoat with nanocrystalline silver in the management of burns. Burns 2004, 30 Suppl 1, S1-9; (c) Argirova, M.; Hadjiski, O.; Victorova, A., Acticoat versus Allevyn as a split-thickness skin graft donor-site dressing: a prospective comparative study. Ann. Plast. Surg. 2007, 59 (4), 415-22.
33. (a) Ibrahim, A., et al., A simple cost-saving measure: 2.5% mafenide acetate solution. J Burn Care Res 2014, 35 (4), 349-53; (b) Studer, N. M., et al., Care of the Burn Casualty in the Prolonged Field Care Environment. J. Spec. Oper. Med. 2015, 15 (3), 86-93; (c) Ahuja, R. B.; Gupta, A.; Gur, R., A prospective double-blinded comparative analysis of framycetin and silver sulphadiazine as topical agents for burns: a pilot study. Burns 2009, 35 (5), 672-6.
34. Paydar, S., et al., A Comparison of the Effects of Topical Prolavacid Solution (a Polyhexamethylene Biguanide-Based Wound Cleanser) and Medihoney Ointment in a Rat Model of Cutaneous Wound. Adv. Wound Care 2017, 6 (12), 407-412.
35. Chindera, K., et al., The antimicrobial polymer PHMB enters cells and selectively condenses bacterial chromosomes. Sci. Rep. 2016, 6 (1), 23121.
36. (a) Malpass, K. G.; Snelling, C. F.; Tron, V., Comparison of donor-site healing under Xeroform and Jelonet dressings: unexpected findings. Plast. Reconstr. Surg. 2003, 112 (2), 430-9; (b) Hansbrough, W.; Dore, C.; Hansbrough, J. F., Management of skin-grafted burn wounds with Xeroform and layers of dry coarse-mesh gauze dressing results in excellent graft take and minimal nursing time. J. Burn Care Rehabil. 1995, 16 (5), 531-4.

1. A method for preparing a temperature-sensitive hydrogel for administration to the skin of a subject in need thereof, comprising aerosol mixing (a) a first solution comprising water and (b) a second solution, to form a hydrogel; wherein
the second solution comprises an organic solvent and a polymer selected from a poly(N-alkylacrylamide) or a polyvinylpyrrolidone copolymer of a first monomer having formula (1) or formula (2) and at least one other monomer that is different than the first monomer: 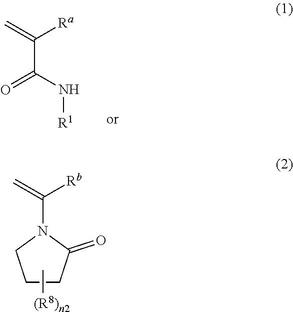
wherein:
R^{a} is H or C₁₋₆ alkyl;
R^{b} is H or C₁₋₆ alkyl;
R¹ is —(CH₂)ₙ₁—R³, C₁₋₆ alkyl, C₆₋₁₈ aryl, or C₄₋₁₈ heteroaryl;
R³ is H, hydroxyl, F, Cl, Br, NH₂, or N(R⁴)₂;
R⁴ is H or C₁₋₆ alkyl;
each R⁸ is independently C₁₋₆ alkyl
n1 is an integer from 0 to 6; and
n2 is 0, 1 or 2; and
optionally wherein the aerosol mixing occurs initially at ambient temperature, and the temperature of the resulting mixture reduces while evaporating of the organic solvent.

2. (canceled)

3. The method of claim 1, further comprising aerosol administration of the hydrogel to the skin of the subject after mixing, and optionally wherein the skin comprises a wound.

4. (canceled)

5. The method of claim 3, wherein the aerosol administration of the hydrogel is performed via an aerosol applicator, and optionally wherein the aerosol applicator comprises an electrospinning (ES) applicator, a solution blown spinning (SBS) applicator, a solution blown deposition (SBD) applicator, or a spray deposition (SD) applicator.

6. (canceled)

7. The method of claim 1, wherein water is present in a mixture of the first solution and the second solution with a weight percentage ranging from about 10% to about 90%, from about 25% to about 75%, or from about 40% to about 60%.

8. (canceled)

9. The method of claim 1, wherein the polymer is present in the second solution with a concentration of about 0 wt % to about 40 wt %, about 5 wt % to about 30 wt %, or about 10 wt % to about 30 wt %.

10. (canceled)

11. The method of claim 1, further comprising evaporating the solvent to form of a fibrous mesh after the aerosol mixing step.

12. The method of claim 3, further comprising administering a fibrous mesh or a polymer support to the skin of subject where the gel is to be administered, optionally the gel is deposited on the skin of subject, over the fibrous mesh, or over a polymer support.

13. (canceled)

14. The method of claim 3, further comprising reducing temperature of the gel to reduce the adhesive strength of the gel on the skin, optionally the temperature of the gel is reduced to less than about 15° C. or less than about 10° C.

15.-16. (canceled)

17. The method of claim 1, wherein the at least one other monomer that is different than the first monomer is described by formula 3a: 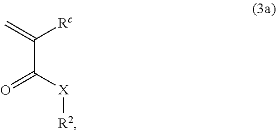
formula 3b: 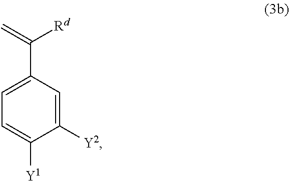
or a combination thereof; wherein
R^{c} is H or C₁₋₆ alkyl;
R^{d} is H or C₁₋₆ alkyl;
R² is H, C₁₋₆ alkyl, C₆₋₁₈ aryl, or C₄₋₁₈ heteroaryl;
X is O or NH; and
Y¹ and Y² are each independently selected from H, C₁₋₆ alkyl, OH, or B(OH)₂.

18. The method of claim 1, wherein the organic solvent is selected from ethyl acetate, acetone, ethanol, and any combination of two or more thereof.

19. A temperature-responsive hydrogel system comprising:
(a) a first solution comprising water, and
(b) a second solution comprising an organic solvent; and
a polymer selected from a poly(N-alkylacrylamide) or a polyvinylpyrrolidone copolymer of a first monomer having formula (1) or formula (2) and at least one other monomer that is different than the first monomer: 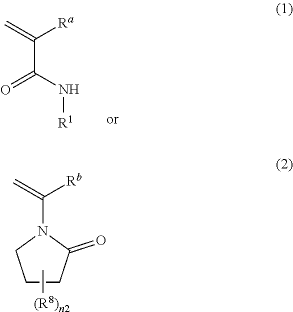
wherein:
R^{a} is H or C₁₋₆ alkyl;
R^{b} is H or C₁₋₆ alkyl;
R¹ is —(CH₂)ₙ₁—R³, C₁₋₆ alkyl, C₆₋₁₈ aryl, or C₄₋₁₈ heteroaryl;
R³ is H, hydroxyl, F, Cl, Br, NH₂, or N(R⁴)₂;
R⁴ is H or C₁₋₆ alkyl;
each R⁸ is independently C₁₋₆ alkyl
n1 is an integer from 0 to 6; and
n2 is 0, 1 or 2.

20.-21. (canceled)

22. The temperature-responsive hydrogel system of claim 19, wherein the at least one other monomer that is different than the first monomer is described by formula 3a: 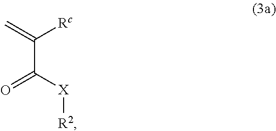
formula 3b: 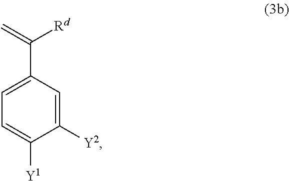
or a combination thereof; wherein
R^{c} is H or C₁₋₆ alkyl;
R^{d} is H or C₁₋₆ alkyl;
R² is H, C₁₋₆ alkyl, C₆₋₁₈ aryl, or C₄₋₁₈ heteroaryl;
X is O or NH; and
Y¹ and Y² are each independently selected from H, C₁₋₆ alkyl, OH, or B(OH)₂.

23-27. (canceled)

28. The temperature-responsive hydrogel system of claim 19, further comprising a cross-linking agent.

29.-33. (canceled)

34. The temperature-responsive hydrogel system of claim 19, further comprising an adhesion-adjusting additive, optionally wherein:
the adhesion-adjusting additive is an adhesion-enhancing additive; or
the adhesion-adjusting additive is a LCST (Lower Critical Solution Temperature)-adjusting additive.

35. (canceled)

36. The temperature-responsive hydrogel system of claim 34, wherein a base temperature-responsive hydrogel having the adhesion-enhancing additive has a failure pressure that is at least 2 times greater than a failure pressure for a base temperature-responsive hydrogel having the same composition without the adhesion-enhancing additive.

37. The temperature-responsive hydrogel system of claim 34, wherein a base temperature-responsive hydrogel having the adhesion-enhancing additive has a failure pressure that is 2 to 6 times greater than a failure pressure for a base temperature-responsive hydrogel having the same composition without the adhesion-enhancing additive.

38.-42. (canceled)

43. The temperature-responsive hydrogel system of claim 34, wherein the LCST-adjusting additive is polyethylene-glycol (PEG).

44.-50. (canceled)

51. The temperature-responsive hydrogel system of claim 19, wherein the polyvinylpyrrolidone copolymer is 3-ethyl-1-vinyl-2-pyrrolidone.

52. (canceled)

53. The temperature-responsive hydrogel system of claim 19, wherein the polyvinylpyrrolidone copolymer is a copolymer formed from monomers comprising 2-ethyl-N-vinylpyrrolidone and butyl acrylate.

54.-55. (canceled)

56. The temperature-responsive hydrogel system of claim 19, further comprising a bioactive agent.

57.-60. (canceled)

61. The temperature-responsive hydrogel system of claim 19, further comprising one or more additional monomers having formula 4 that are different than the first monomer and second monomer: 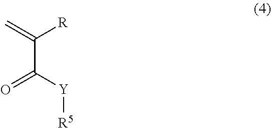
wherein:
Y is O or NR⁶;
R is H or C₁₋₆ alkyl;
R⁵ is —(CH₂)ₘ—R₇;
R⁶ is H or C₁₋₆ alkyl;
R⁷ is halo, hydroxyl, C₆₋₁₂ aryl, C₄₋₁₈ heteroaryl, amino, phosphorylcholinyl, or pyridinyl; and
m is an integer from 0 to 18.

62. A fibrous mesh or a uniform gel film formed from the temperature-responsive hydrogel system of claim 19.

63. (canceled)

64. The fibrous mesh or the uniform gel film of claim 62, having an adhesive strength sufficient for keeping the fibrous mesh or the uniform gel film in place during subject movement at skin temperature.

65.-67. (canceled)

68. A method for preparing a temperature-responsive or temperature-sensitive hydrogel, comprising aerosol mixing of the first solution and the second solution of claim 19, to form a hydrogel.

69. (canceled)

70. A method for treating the skin of a subject in need thereof comprising applying the temperature-sensitive hydrogel of claim 19 to form a hydrogel, thereby treating the subject.

71.-75. (canceled)

76. A kit comprising the system of claim 19, and instructions for use.
